SEQUENCE LISTING (3 sequences)

SEQ ID NO: 1 - protein, 10 aa, Mus musculus
  MISC_FEATURE (1)..(10): Chromogranin A (CgA) Peptide
YVRPLWVRME

SEQ ID NO: 2 - protein, 17 aa, Mus musculus
  MISC_FEATURE (1)..(17): E-alpha Peptide
ASFEAQGALANIAVDKA

SEQ ID NO: 3 - protein, 9 aa, Mus musculus
  MISC_FEATURE (1)..(9): NRP-V7 Peptide
KYNKANVFL

(19) United States
(12) Patent Application Publication
(10) Pub. No.: US 2024/0245762 A1
(43) Pub. Date: Jul. 25, 2024

(54) METHODS AND PLATFORMS FOR PROMOTING ANTIGEN-SPECIFIC TOLERANCE IN THE TREATMENT OF TYPE 1 DIABETES AND GRAFT REJECTION AND COMPOSITIONS RELATING THERETO

(71) Applicants: University of Maryland, College Park, College Park, MD (US); University of Maryland, Baltimore, Baltimore, MD (US)

(72) Inventors: Christopher M. JEWELL, Silver Spring, MD (US); Joshua M. GAMMON, Cambridge, MA (US); Lisa H. TOSTANOSKI, Boston, MA (US); Jonathan BROMBERG, Baltimore, MD (US)

(21) Appl. No.: 18/261,203

(22) PCT Filed: Jan. 12, 2022
(86) PCT No.: PCT/US2022/012100

§ 371 Date: Jul. 12, 2023

Related U.S. Application Data
(60) Provisional application No. 63/265,571, filed on Dec. 16, 2021.
(60) Provisional application No. 63/137,091, filed on Jan. 13, 2021.

(51) Int. Cl. A61K 39/00; A61K 31/436; A61P 37/06

(52) U.S. Cl. CPC A61K 39/0008; A61K 31/436; A61P 37/06; A61K 2039/55555; A61K 2039/577

(57) ABSTRACT

The present disclosure is directed to systems, compositions and methods for promoting immune tolerance to an antigen in a subject. Systems and methods provide for introducing directly into at least one lymph node(s) of the subject a therapeutically effective amount of a composition comprising an antigen associated with an autoimmune disease or disorder, in combination with a carrier comprising an immune modulatory agent such that an immune response to said antigen is inhibited or suppressed in the subject. The present disclosure is also directed to systems, compositions and methods for the treatment and/or prevention of autoimmune diseases and conditions, and in particular type 1 diabetes and graft rejection.

### CROSS-REFERENCE TO RELATED APPLICATIONS

This application is based on U.S. Provisional Patent Application Ser. No. 63/137,091, entitled “System, Materials, and Methods for Intra-Lymph Node Delivery of Microparticle Depots,” filed Jan. 13, 2021, and U.S. Provisional Patent Application Ser. No. 63/265,571, entitled “Systems, Materials, and Methods for Intra-Lymph Node Delivery of Microparticle Depots,” filed Dec. 16, 2021, which applications hereby are incorporated herein by reference in their respective entireties and to which priority is claimed.

### STATEMENT REGARDING FEDERALLY SPONSORED RESEARCH OR DEVELOPMENT

This invention was made with government support under Grant No. R01EB026896 awarded by the National Institute of Health (NIH). The United States government has certain rights in this invention.

### FIELD OF DISCLOSURE

The present disclosure relates to methods for promoting tolerance to an antigen(s) associated with an autoimmune disease or condition, and, in particular, type 1 diabetes and graft rejection, via intra-lymph node delivery of the antigen(s) and/or an immune modulatory agent for the treatment or prevention of such autoimmune disease, or condition. The present disclosure also concerns pharmaceutical compositions that comprise a therapeutically effective amount of an antigen and/or an immune modulatory agent capable of inhibiting or suppressing an immune response in a subject for the treatment of an autoimmune disease or condition. The disclosure also relates to the use of such pharmaceutical compositions in the treatment or prevention of an autoimmune disease or condition in a recipient subject.

### BACKGROUND

The body's immune system serves as a defense against a variety of conditions, and is mediated by two separate but interrelated systems: the cellular and humoral immune systems.

Generally, the humoral immune system is mediated by soluble products (antibodies or immunoglobulins) that have the ability to combine with and neutralize structures recognized by the system as being foreign to the body. The cellular immune system involves the mobilization of certain cells (T cells) that serve a variety of therapeutic roles. T cells are lymphocytes that are derived from the thymus and circulate between the tissues, lymphatic system, and circulatory system. They act against, or in response to, a variety of foreign structures (antigens). Although T cells do not themselves secrete antibodies, they are usually required for antibody secretion by the second class of lymphocytes, B cells (which derive from bone marrow). As known in the art, T cells exhibit extraordinary immunological specificity so as to be capable of discerning one antigen from another.

A naive T cell, e.g., a T cell which has not yet encountered its specific antigen, is activated when it first encounters a specific peptide:MHC complex on an antigen presenting cell (APC). The APC may be a B cell, a macrophage or a dendritic cell. When a naive T cell encounters a specific peptide:MHC complex on an APC, a signal is delivered through the T-cell receptor which induces a change in the conformation of the T cell's lymphocyte function associated antigen (LFA) molecules, and increases their affinity for intercellular adhesion molecules (ICAMs) present on the surface of the APC. The signal generated by the interaction of the T cell with an APC is necessary, but not sufficient, to activate a naive T cell. A second co-stimulatory signal is required. The naive T cell can be activated only by an APC carrying both a specific peptide MHC complex and a co-stimulatory molecule on its surface. Antigen recognition by a naive T cell in the absence of co-stimulation results in the T cell becoming anergic. The need for two signals to activate T cells and B cells such that they achieve an adaptive immune response may provide a mechanism for avoiding responses to self-antigens that may be present on an APC at locations in the system where it can be recognized by a T cell. Where contact of a T cell with an APC results in the generation of only one of two required signals, the T cell does not become activated and an adaptive immune response does not occur.

The efficiency with which humans and other mammals develop an immunological response to pathogens and foreign substances rests on two characteristics: the specificity of the immune response for antigen recognition, and the immunological memory that allows for faster and more vigorous responses upon re-activation with the same antigen (Portoles, P. et al. (2009) “The TCR CD3 Complex: Opening the Gate to Successful Vaccination,” Current Pharmaceutical Design 15:3290-3300; Guy, C. S. et al. (2009) “Organization of Proximal Signal Initiation at the TCR:CD3 Complex,” Immunol Rev. 232(1):7-21). The specificity of the response of T-cells is mediated by the recognition of antigen (displayed on APCs) by a molecular complex involving the T Cell Receptor (“TCR”) and the cell surface receptor ligand, CD3. The complex is particularly significant since it contains a large number of immunoreceptor tyrosine-based activation motifs (ITAMs).

Thus, the adaptive immune response provides antigen-specific protection against foreign pathogens while preserving tolerance against self-tissue. However, tolerance can break down resulting in autoimmune disease such as type 1 diabetes (T1D), where pancreatic islets are targeted and destroyed. Pancreatic islets are small islands of cells that produce hormones that regulate blood glucose levels. Hormones produced in the pancreatic islets are secreted directly into the blood by five different types of cells: alpha cells that produce glucagon; beta cells that produce insulin and amylin; delta cells that produce somatostatin; gamma cells that produce pancreatic polypeptide; and epsilon cells that produce ghrelin. While T1D can be managed with frequent insulin administration, multiple comorbidities exist including cardiovascular and gastrointestinal disease (Schofield, J. et al. (2019) Cardiovascular Risk in Type 1 Diabetes Mellitus. Diabetes Ther. 10, 773-789; Khoshbaten, M. et al. (2011) Gastrointestinal signs and symptoms among persons with diabetes mellitus. Gastroenterol. Hepatol. Bed Bench 4, 219-223). There are currently no approved and/or effective therapies targeting the underlying immunopathology of T1D.

Existing clinical therapies for autoimmune disease aim to restrain inflammation and self-reactivity using systemic administration of immunosuppressive drugs or modulatory therapies (Fugger, L. et al. (2020) Challenges, Progress, and Prospects of Developing Therapies to Treat Autoimmune Diseases. Cell 181, 63-80). These treatments are not curative and require life-long compliance. Even the newest monoclonal antibodies do not distinguish between healthy and self-reactive cells, leading to serious side effects and non-specific immunosuppression (Hansel, T. T. et al. (2010) The safety and side effects of monoclonal antibodies. Nat. Rev. Drug Discov. 9, 325-338). For example, a monoclonal antibody targeting CD3, which is thought to preferentially deplete effector CD8 T cells (Masharani, U. B. & Becker, J. (2010) Teplizumab therapy for type 1 diabetes. Expert Opin. Biol. Ther. 10, 459-465), has delayed the loss of insulin production in patients previously diagnosed with T1D and was recently demonstrated as the first immunotherapy in a clinical trial to delay disease progression in at risk T1D patients (Herold, K. C. et al. (2019) An Anti-CD3 Antibody, Teplizumab, in Relatives at Risk for Type 1 Diabetes. N. Engl. J. Med. 381:603-613; Sims, E. K. et al. (2021) Teplizumab improves and stabilizes beta cell function in antibody-positive high-risk individuals. Sci. Transl. Med. 13, eabc8980). While more specific than conventional immunosuppressants, this treatment is not curative and does not have the ability to distinguish between healthy and autoreactive T cells.

Another therapy for T1D involves transplantation of allogeneic islets to restore control of insulin, yet these grafts are non-self, resulting in attack by recipient (i.e., host) T and B lymphocytes (Cayabyab, F., Nih, L. R. & Yoshihara, E. (2021) Advances in Pancreatic Islet Transplantation Sites for the Treatment of Diabetes. Front. Endocrinol. 12, 1077; Ricordi, C. et al. (1992) Human islet isolation and allotransplantation in 22 consecutive cases. Transplantation 53, 407-414). Thus, simultaneous broad immunosuppression is required in an attempt to slow graft rejection (Nakamura, T. et al. (2020) Long-term outcome of islet transplantation on insulin-dependent diabetes mellitus: An observational cohort study. J. Diabetes Investig. 11, 363-372).

Another experimental therapeutic concept is the induction of tolerance against specific self-antigens attacked in autoimmune disease, without inhibiting normal adaptive immune responses needed to fight infection (Gammon, J. M. & Jewell, C. M. (2019) Engineering Immune Tolerance with Biomaterials. Adv. Healthc. Mater. 8, 1801419; Gosselin, E. A. et al. (2018) Designing natural and synthetic immune tissues. Nat. Mater. 17, 484-498). This strategy involves administration of vaccine-like treatments co-delivering relevant autoantigens with regulatory immune cues to promote autoantigen-specific T cells with regulatory immune function (Froimchuk, E. et al. (2021) Biophysical Properties of Self-Assembled Immune Signals Impact Signal Processing and the Nature of Regulatory Immune Function. Nano Lett. 21, 3762-3771; Oakes, R. S. et al. (2021) Exploiting Rational Assembly to Map Distinct Roles of Regulatory Cues during Autoimmune Therapy. ACS Nano 15, 4305-4320; Tostanoski, L. H. et al. (2019) Engineering release kinetics with polyelectrolyte multilayers to modulate TLR signaling and promote immune tolerance. Biomater. Sci. 7, 798-808; Tostanoski, L. H. et al. (2016) Design of Polyelectrolyte Multilayers to Promote Immunological Tolerance. ACS Nano 10, 9334-9345; Maldonado, R. A. et al. (2015) Polymeric synthetic nanoparticles for the induction of antigenspecific immunological tolerance. Proc. Natl. Acad. Sci. 112, E156-E165; Saito, E. et al. (2020) Modulating lung immune cells by pulmonary delivery of antigen-specific nanoparticles to treat autoimmune disease. Sci. Adv. 6, eabc9317; Kenison, J. E. et al. (2020) Tolerogenic nanoparticles suppress central nervous system inflammation. Proc. Natl. Acad. Sci. U.S.A 117, 32017-32028; Yeste, A. et al. (2016) Tolerogenic nanoparticles inhibit T cell-mediated autoimmunity through SOCS2. Sci. Signal. 9, ra61). A number of these approaches focus on generation of regulatory T cells (T_{REG}), which exhibit potent tolerizing capabilities (Sharabi, A. et al. (2018) Regulatory T cells in the treatment of disease. Nat. Rev. Drug Discov. 17, 823-844). Because the fate of T cells during antigen encounter depends on signal integration in lymph nodes (LNs), tissues that coordinate immunity (Jain, A. & Pasare, C. (2017) Innate Control of Adaptive Immunity: Beyond the Three-Signal Paradigm. J. Immunol. 198, 3791-3800), a challenge of this approach is ensuring that self and regulatory cues are presented in appropriate combinations and concentrations in LNs.

Thus, immunotherapies for autoimmune disease and allograft rejection aim to promote antigen-specific tolerance. These treatments seek to selectively inhibit detrimental immune response without compromising functional protective immunity. However, a major obstacle to conventional immunotherapies is ineffective targeting of lymph nodes, where adaptive immune responses develop. Novel treatments which offer molecular specificity are needed to address the persistent and difficult challenges and disadvantages of existing methodologies.

### SUMMARY OF THE DISCLOSURE

The present disclosure relates to systems, compositions, and methods for intra-lymph node delivery of microparticles. In some embodiments, the systems, compositions, and methods provide for controlled delivery of a therapeutic agent, e.g., regulatory immune cues and/or peptide antigens. In some embodiments, the systems, compositions, and methods provide for size-restricted polymer carriers or scaffolds coupled to or encapsulating a therapeutic agent, e.g., regulatory immune cues and/or peptide antigens. In some embodiments, the disclosed systems, compositions and methods are utilized for the treatment and/or prevention of an autoimmune disease or condition, and in particular type 1 diabetes (T1D) or graft transplant rejection.

In some embodiments, methods of treating or preventing an autoimmune system provide for the introduction into at least one lymph node (LN) of a subject, e.g., via intra-lymph node injection, biodegradable microparticles encapsulating a relevant antigen(s), and an immunosuppressive agent or molecule, such as rapamycin. As demonstrated, the methods of the present disclosure potently inhibited disease in models of T1D and allogenic islet transplantation. The use of both antigen and an immunosuppressive agent (e.g., rapamycin) provided for optimal efficacy, with microparticle induced tolerance accompanied by expansion of antigen-specific regulatory T cells in treated and untreated lymph nodes. Further, microparticle treatment resulted in the development of tolerogenic structural microdomains in lymph nodes. The microparticle treatment also promoted expression of memory markers among antigen-specific regulatory T cells, and durable tolerance in T1D. As demonstrated herein, intra-lymph node injection of tolerogenic microparticles may be utilized as a powerful strategy to promote tolerance in the treatment and/or prevention of various autoimmune diseases and conditions, and in particular T1D and graft transplant rejection.

The present disclosure relates to a method of promoting immune tolerance to an antigen in a subject. The method comprises introducing directly into at least one lymph node of the subject: a therapeutically effective amount of a composition comprising an antigen associated with an autoimmune disease or disorder, in combination with a carrier comprising an immune modulatory agent such that an immune response to said antigen is inhibited or suppressed in the subject. The present disclosure also relates to a method of treating or preventing an autoimmune disease or condition in a subject in need thereof, comprising introducing directly into at least one lymph node of the subject: a therapeutically effective amount of a composition comprising an antigen associated with an autoimmune disease or disorder, in combination with a carrier comprising an immune modulatory agent such that an immune response to said antigen is inhibited or suppressed in the subject. In some embodiments, the antigen and/or the immune modulatory agent is introduced directly into the at least one lymph node of the subject via intra-lymph node injection. In preferred embodiments, the subject is a human.

The present disclosure also relates to a pharmaceutical composition comprising a therapeutically effective amount of an antigen associated with an autoimmune disease or disorder, an immune modulatory agent formulated to inhibit or suppress an immune response to the antigen in a subject, and a pharmaceutically acceptable carrier, diluent, and/or excipient. In preferred embodiments, the pharmaceutical composition is formulated for administration via intra-lymph node injection.

In some embodiments, the carrier is a microparticle. In some embodiments, the microparticle has a diameter of between about 1 μm and about 10 μm. Preferably, the microparticle is biodegradable and/or biocompatible. In some embodiments, the microparticle comprises a polymer material selected from the group consisting of poly(glycolide) (PGA), poly(L-lactide) (PLA), poly(beta-amino esters), and polyethylene glycol (PEG).

In some embodiments, the immune modulatory agent is selected from the group consisting of a calcineurin inhibitor, an interleukin inhibitor, a TNF alpha inhibitor, and a selective immunosuppressant. In some embodiments, the immune modulatory agent is an mTOR inhibitor. In some embodiments, the mTOR inhibitor is rapamycin or derivative thereof.

In some embodiments, the antigen and the immune modulatory agent are introduced into a single lymph node of the subject. In some embodiments, the antigen and the immune modulatory agent are introduced into at least two lymph nodes of the subject. In some embodiments, the antigen is introduced into one or more lymph node(s) of the subject, and the immune modulatory agent is introduced into one or more lymph node(s) of the subject different from the one or more lymph node(s) into which the antigen is introduced.

In some embodiments, the autoimmune disease or condition is type 1 diabetes. In some embodiments, the autoimmune disease or condition is associated with a graft transplant. In some embodiments, the antigen is an islet antigen. In some embodiments, the antigen is selected from the group consisting of: chromogranin A (CgA); NRP-V7; insulin; GAD65; GAD67; carboxypeptidase H; insulinoma associated antigen-2 (IA-2); insulinoma associated antigen-2 beta (IA-2β; also termed phogrin or ICA512); imogen 38; islet-specific glucose-6-phosphatase catalytic subunit-related protein (IGRP); ZnT8: islet amyloid polypeptide (IAPP); Tetraspanin-7 (Tspan7); P4Hb; GRP78; Urocortin-3; an d MHC antigen class I and class II molecules (including Ea and E$).

### BRIEF DESCRIPTION OF THE DRAWINGS

FIG. 1. (a) Onset of T1D following transfer of islet-specific cells as measured by blood glucose. Fraction indicates proportion surviving at termination of study. (b) Survival plots of islet transplant grafts following different intra-LN MP treatments, with median survival indicated by each treatment.

FIG. 2. (a) Representative flow cytometry plots of proliferated T cells by treatment type measured by CFSE proliferative dye dilution. Thy1.1 staining indicates an injected, antigen-specific cell versus a native T cell. Numbers indicate percent of parent. (b) Proportion of antigen-specific cells that are forkhead box P3 (FoxP3)/CD25+ (T_{REG}) at different LN locations following treatment. (c) Representative flow cytometry plots of Foxp3 expression against Eα-specific T cells by particle type. Number indicates percent of parent.

FIG. 3. (a-b) Lama4/Lama5 ratios in LNs treated with each MP type for T1D in both the (a) high endothelial venules (HEV) and the (b) cortical ridge (CR). (c-d) Lama4/Lama5 ratios in the (c) HEV and the (d) CR for the islet transplantation model. As shown, CgA/rapamycin MPs promote increased Lama4/Lama5 ratios in HEVs and CRs. (e-f) Changes in Foxp3 colocalization in HEVs and CR were not seen in LNs treated with CgA/rapamycin MPs, which did not promote local accumulation of T_{REGs} in HEVs and CRs.

FIG. 4. (a) Representative flow plots showing CD62L+CD44+ expression among Foxp3 positive T_{REG} cells treated with both antigen and antigen-rapamycin MPs. (b) Proportion of CD62L+CD44+ T_{REG} cells among cells treated with different MP groups and lipopolysaccharide (an immune activating signal).

FIG. 5. MPs encapsulating relevant antigens and rapamycin promote suppressive T cell responses in vitro. BDC2.5 T cell co-cultures were treated with CgA MP or CgA/rapamycin MPs. (a) Quantification of BDC2.5 T cell proliferation and (b) representative flow cytometry plots of carboxyfluorescein succinimidyl ester (CFSE) dilution are shown. (c) quantification of Foxp3 expression and (d) representative flow cytometry plots are shown. (e) quantification of IFNγ expression and (f) representative flow cytometry plots are shown. Eα T cell co-cultures were treated with Eα MP or Eα/rapamycin MPs. Quantification of (i) proliferation, (j) Foxp3 expression, and (k) IFNγ expression are shown. NRP-V7 T cell co-cultures were treated with NRP-V7 MP or NRP-V7/rapamycin MPs. (1) representative flow cytometry plots showing proliferation and quantification of (m) proliferation and (n) IFNγ secretion are shown. N=3 individual culture wells for all experiments. Plots represent mean s.d. and one way ANOVA with Tukey's post testing were used to compare treatment groups and comparisons between matched doses of CgA and CgA/rapamycin MPs are shown.

FIG. 6. Gating scheme for T cell co-culture studies with (a) CgA and Eα specific T cells and (b) NRP-V7 specific T cells detailed in FIG. 5.

FIG. 7. Intra-LN injection of tolerogenic MPs promotes tolerance in T1D and suppression of graft rejection in allogenic islet transplantation. (a) Schematic representation of intra-LN treatment in the NRP-V7 model of T1D. (b) Survival curve with number of mice which developed disease out of the total mice per group indicated in the parenthesis in the legend. (c) Individual blood glucose traces for mice in each group from (b). (d) Schematic representation of intra-LN treatment in the non-matched islet transplant model. (e) Survival curve for experiment detailed in (d). (f) Individual blood glucose traces for mice in each group (e). N=5 mice for all groups except N=4 mice for the rapamycin MP treatment group. Two tailed log-rank (Mantel-Cox) was used for all pairwise comparisons between each treatment for survival curves in (b) and (e).

FIG. 8. CgA/rapamycin MPs promote antigen-specific T cell priming while inhibiting mTOR in vivo. (a) Schematic representation of MP treatment and experimental readout for BDC2.5 T cell adoptive transfer experiment. (b) Illustration of lymph nodes collected and their anatomical relevance to treatment. (c) Representative flow cytometry traces and (d) quantification of CD69 expression among CgA specific T cells. (e) Representative flow cytometry traces of phosphorylated s6 among CD69+CgA specific T cells in treated LNs and (f) quantification in all LNs. N=4 mice for all experiments, and paired LNs were pooled for each mouse. The mean±s.d. is shown. One way ANOVA with Tukey's post hoc testing was used to compare each treatment within individual lymph nodes.

FIG. 9. (a) Gating scheme to analyze CD69 and ps6 expression among CgA specific T cells in the experiment detailed in FIG. 8. (b) Geometric mean fluorescence intensity of ps6 among CgA specific CD69⁺ cells.

FIG. 10. CgA/rapamycin MPs expand antigen-specific Treg in treated and untreated LNs. (a) Schematic representation of MP treatment and experimental readout for BDC2.5 T cell adoptive transfer experiment. (b) Illustration of LNs collected and analyzed. (c) Representative flow cytometry traces and quantification of CgA specific T cell proliferation at (d) day 2 and (e) day 7. Quantification of CgA specific T cell frequency at (f) day 2 and (g) day 7. Representative flow cytometry traces showing foxp3 expression among CgA specific T cells in treated LNs at (h) day 2 and (i) day 7. Quantification of foxp3 expression among CgA specific T cells at (j) day 2, and (k) day 7 in each LN analyzed. N=5 mice for all groups in all experiments, and paired LNs were pooled for each mouse. The mean s.d. is shown. One way ANOVA with Tukey's post hoc testing was used to compare each treatment within individual lymph nodes.

FIG. 11. Gating scheme to analyze Foxp3 expression and CFSE dilution among CgA specific T cells in the experiments detailed in FIG. 10.

FIG. 12. Eα is presented in treated and untreated LNs after Ea/rapamycin MP treatment. (a) Schematic representation of experiment in panels b-d. N=3 mice for DiR MP and FITC-CgA/DiR MP treatment groups and N=1 mouse for empty MP treatment group at each timepoint. Each data point is represented as an individual data point. (b) IVIS images of excised LNs in treated LNs. (c) quantitation of MP signal and (d) Cy5-CgA signal in excised LNs. One way ANOVA with Tukey's post hoc testing was used to compare each treatment group, at each time point. Individual LNs were used as single data points. Mean±s.d. is shown. (e) Schematic representation of experiment in panels f-i. N=2 mice for each timepoint. (f) Representative immunofluorescent images of LNs stained with ER-TR7, CDT Ic, YAe; Scale bar=50 μm. Quantification of Eα presentation in MHC-II (YAe) in (g) treated, (h) early-draining and (i) late-draining LNs. One way ANOVA with Tukey's post hoc testing was used to compare each indicated LNs at each timepoint. j) Schematic representation of experiment in panels k-m. Quantification of Eα presentation in MHC-II in (k) treated, (1) early-draining and (m) late-draining LNs after FTY720 treatment. One way ANOVA with Tukey's post hoc testing was used to compare each group. Mean±s.d. is shown for each sample in each experiment.

FIG. 13. MP treatment promotes development of tolerogenic microdomains in LNs. (a) Schematic representation of experiment in panels b-d. N=4 mice for each treatment group. (b) Representative immunofluorescence images of treated LNs stained for laminin α4, laminin α5, ER-TR7 and DAPI are shown; Scale bar=50 μm. Quantification of the ratio of laminin α4 to laminin α5 in (c) high endothelial venules and (d) cortical ridges in treated LNs. One way ANOVA with Tukey's post hoc testing was used to compare treatment groups. (e) Schematic representation of experiment in panel f. N=4 mice for each treatment group. Inguinal, axillary, pancreatic and popliteal LNs were pooled for each treatment group. (f) Ratio of laminin α4 to laminin α5 in high endothelial venules of LNs. Two tailed Mann Whitney testing was used to compare both treatment groups. (g) Schematic representation of experiments in panels i-l. N=4 mice for each treatment group. (h) Representative immunofluorescence images showing Foxp3 expression among antigen-specific T cells. DAPI (Blue) and ER-TR7 (Cyan) are included in the merge: Scale bar=50 μm. LNs were stained for Foxp3, Vα4 T cell receptor clonotype, ER-TR7, and DAPI. Quantification of Foxp3 expression among antigen-specific T cells in (i) high endothelial venules and (j) cortical ridges. One way ANOVA with Tukey's post hoc testing was used to compare each treatment group within individual lymph nodes in each region of the LN analyzed. For all experiments mean±s.d. is shown.

FIG. 14. MP treatment promotes expression of memory markers among antigen-specific Treg and durable tolerance in T1D. BDC2.5 T cell co-cultures were treated with CgA MPs or CgA/rapamycin MPs. (a) representative gating scheme to quantify co-expression of CD44 and CD621 among BDC2.5 Treg. Quantification of (b) co-expression of CD44 and CD62L among BDC2.5 Treg and (d) mean fluorescence intensity of CCR7 among BDC2.5 Treg. N=3 for each sample. One way ANOVA with Tukey's post hoc testing was used to compare all groups. Comparisons for CgA MP and CgA/rapamycin MPs at matched doses are indicated in plots. (e) Schematic representation of BDC2.5 T cell adoptive transfer experiment in panels f-k. N=5 for each treatment group. (f) Representative flow cytometry plots showing BDC2.5 T cell frequency in treated LNs. Gating scheme for (g) CgA specific Treg and (h) co-expression of CD44 and CD621, and expression of BCL-2 and CD127 among CgA specific T_{REG}. Quantification of (i) co-expression of CD44 and CD621, and expression of (j) BCL-2 and (k) CD127 among CgA specific T_{REG} in treated LNs. Treated LNs were pooled for each mouse. Two tailed Mann Whitney testing was used to compare CgA MP vs CgA/rapamycin MP treatment groups. (1) Schematic representation of NRP-V7 adoptive transfer induced T1D experiment. N=8 for each treatment group. The number of surviving mice in each treatment group is shown in parenthesis in the legend. (m) Survival curve indicating percent of mice remaining normoglycemic. Two tailed log-rank (Mantel-Cox) was used for all pairwise comparisons between each treatment.

FIG. 15. Schematic illustrations of the disclosed platform of the present disclosure showing: (a) immune suppression of non-matched islet transplant rejection via intra-LN injection of carriers containing rapamycin and Eα; and (b) immune tolerance in T1D via intra-LN injection of carriers containing rapamycin and CgA and NRP-V7.

FIG. 16. Autoantigen/rapamycin MPs promote a suppressive T cell response in vitro during DC-T cell co-cultures. DC/T cell co-cultures treated with relevant auto/alloantigen+Rapa MPs were conducted using TCR transgenic T cells. (a-c) Antigen/rapamycin MPs are modular since they can incorporate different antigens and function as expected at suppressing T cell responses of different specificities in vitro. (a-c) Ea/rapamycin MPs inhibited T cell proliferation, increased T_{REG}, and decreased inflammatory cytokine secretion and in vitro. (d-f) NRP-V7/rapamycin MPs inhibited T cell proliferation and decreased inflammatory cytokine secretion in vitro.

FIG. 17. CgA/rapamycin MPs initially inhibited suppressed T cell response compared to treatment with CgA MPs (a) at day 2, but T cell proliferation recovered (b) at day 7. (c) Representative CFSE plots. (d-e) At day 7 post transfer, frequency of transferred T cells in LNs is increased after CgA/rapamycin MP treatment. (f-g) 7 days post transfer, CgA/rapamycin MP treatment increases Foxp3 expression among CgA-specific T cells.

FIG. 18. Ea/rapamycin MPs inhibit activation of Eα specific T cells and increase foxp3 expression in grafts. BALB/c islets were transplanted underneath the renal capsule of C57BL/6 diabetic recipients. At the time of transplantation, recipients were given empty-MP, Eα MP, rapamycin MP or Ea/rapamycin MP by intra-LN injection and TEa cells by i.v. injection. 7 days later, LNs, grafts, and spleens were analyzed with flow cytometry for the activation of Tea cells by CD44 and CD69 expression. Intra-LN injection antigen/rapamycin MPs inhibited T cell effector function at sites of disease. Ea/rapamycin MPs inhibited Eα specific T cell activation in islet grafts, and both CgA/rapamycin MPs and NRP-V7/rapamycin MPs inhibited inflammatory function of NRP-V7 T cells in pancreatic islets.

### DETAILED DESCRIPTION OF EMBODIMENTS

The present disclosure encompasses methods, compositions, and systems utilizing the direct deposit or introduction of therapeutically effective amounts of an antigen(s) and an immune modulatory agent(s) into one or more lymph node(s) of a subject for treating and/or preventing an autoimmune disease or condition, and in particular T1D or graft rejection. Preferably, such introduction of antigen(s) and agent(s) into the LN(s) provides for direct intra-LN injection.

In some embodiments, the immune modulatory agent is rapamycin, an inhibitor of the mechanistic target of rapamycin (mTOR) pathway, or a rapamycin derivative (rapalog), including but not limited to, everolimus, temsirolimus, and sirolimus. Further, other suitable immune modulatory agents may be utilized in accordance with the present disclosure. For example, suitable immune modulatory agents include, but are not limited to, a calcineurin inhibitor (e.g., cyclosporine, tacrolimus, and pimecrolimus), an interleukin inhibitor (e.g., IL-1, IL-2, IL-3, IL-4, IL-5, IL-6, IL-8, IL-17 inhibitor), a tumor necrosis factor (TNF)-alpha inhibitor (e.g., etanercept, infliximab, adalimumab, certolizunab pegol, and golimumab), and/or any other selective immunosuppressant(s). Any such immune modulatory agents known in the art may be utilized in accordance with disclosed embodiments.

In accordance with the present disclosure, the target or selected antigen(s) may be an antigen associated with an autoimmune disease or condition including, but not limited to, chromogranin A (CgA); NRP-V7; insulin; GAD65; GAD67; carboxypeptidase H; insulinoma associated antigen-2 (IA-2); insulinoma associated antigen-2 beta (IA-2β; also termed phogrin or ICA512); imogen 38; islet-specific glucose-6-phosphatase catalytic subunit-related protein (IGRP); ZnT8; islet amyloid polypeptide (IAPP); Tetraspanin-7 (Tspan7); P4Hb; GRP78; Urocortin-3; and MHC antigens (e.g., Eα and Eβ) including class I and class II molecules. Other suitable antigens associated with autoimmune diseases and disorders, and in particular T1D and graft rejection, are known in the art.

As used herein, the terms “treatment” or “treating” refers to an approach for obtaining a beneficial or desired result, and preferably a beneficial or desired clinical result. Such beneficial or desired clinical result includes, but is not limited to, one or more of the following: suppressing or inhibiting an immune response of the subject, decreasing a symptom resulting from the disease, increasing the quality of life of those suffering from the disease, decreasing the dose of other medications required to treat the disease, enhancing the effect of another medication such as via targeting and/or internalization, delaying the progression of the disease, and/or prolonging survival of an individual. As used herein, a “suppressed” immune response means a weakened or diminished immune response, e.g., to a particular antigen. As used herein, an “inhibited” immune response means a prevented or blocked immune response, e.g., to a particular antigen. Subjects for treatment include animals, most preferably mammalian species such as non-primate (e.g., bovine, equine, feline, canine, rodent, etc.) or primate (e.g., monkey, such as, cynomolgus monkey, human, etc.). In preferred embodiments, the subject is a human.

In accordance with disclosed embodiments, a therapeutically effective amount of an antigen and/or an immune modulatory agent is administered to a subject in need thereof, such that an immune response to the antigen is inhibited, suppressed or decreased. The present disclosure also relates to pharmaceutical compositions that comprise a therapeutically effective amount of an antigen and/or an immune modulatory agent capable of altering an immune response, and more preferably inhibiting or suppressing the immune system response against the antigen. The disclosure also relates to the use of such pharmaceutical compositions to treat and/or prevent autoimmune disorders, and, in particular, T1D or graft transplant rejection.

As used herein, the term “an effective amount” of an ingredient (e.g., a molecule, compound, drug, or other agent), or of a pharmaceutical composition or formulation, refers to an amount sufficient to effect beneficial or desired results including, without limitation, clinical results such as decreasing symptoms resulting from the disease attenuating a symptom of an autoimmune disease or disorder, thereby increasing the quality of life of those suffering from the disease, decreasing the dose of other medications required to treat the disease, enhancing the effect of another medication such as via targeting and/or internalization, delaying the progression of the disease, and/or prolonging survival of individuals. When applied to a single active ingredient, the term refers to that ingredient alone. When applied to a combination, the term refers to combined amounts of the active ingredients that result in the therapeutic effect, whether administered in combination, serially, or simultaneously. An effective amount can be administered in one or more administrations and/or in one or more formulations or compositions. While individual needs vary, determination of optimal dosage ranges of effective amounts of an ingredient or composition is within the skill of the art. Empirical considerations, such as the biological half-life, also generally contribute to the determination of the dosage.

As used herein, the term “agent” refers to a biological, pharmaceutical, or chemical compound. Non-limiting examples include simple or complex organic or inorganic molecules, a polypeptide or peptide, a protein, an oligonucleotide, an antibody, an antibody derivative or fragment, a vitamin derivative, a carbohydrate, a toxin, or a chemotherapeutic compound. Agents that are employed in the disclosed methods and compositions can be randomly selected or rationally selected or designed.

The terms “polypeptide,” “oligopeptide,” “peptide” and “protein” are used interchangeably herein to refer to polymers of amino acids of any length, but especially lengths greater than 5, 10, 15, 20 or 25 amino acid residues. The polymer may be linear or branched, it may comprise modified amino acids, and it may be interrupted by non-amino acids. The terms also encompass an amino acid polymer that has been modified naturally or by intervention.

### Pharmaceutical Compositions

Where more than one ingredient or therapeutic agent is to be administered, the antigen(s) and an immune modulatory agent(s) may be formulated together in the same formulation or composition, or may be formulated into separate compositions. Accordingly, in some embodiments, an antigen(s) and an immune modulatory agent(s) are formulated together in the same pharmaceutical composition. In alternative embodiments, an antigen(s) is formulated in one formulation or pharmaceutical composition, and an immune modulatory agent(s) is formulated in another separate formulation or pharmaceutical composition.

The pharmaceutical compositions of the present disclosure may include auxiliary ingredients or components such as pharmaceutically acceptable carriers, diluents and/or excipients. As used herein, the term “pharmaceutically acceptable” means approved by a regulatory agency of the Federal or a state government or listed in the U.S. Pharmacopeia or other generally recognized pharmacopeia for use in animals, and more particularly in humans. Such auxiliary ingredients are selected to not affect the biological activity of the active ingredients or agents. Such auxiliary ingredients are well known in the art and are relatively inert substances that facilitate administration of a pharmacologically effective substance or which facilitate processing of the active compounds into preparations that can be used pharmaceutically for delivery to the site of action.

In addition, the pharmaceutical composition or formulation may also include other carriers, or non-toxic, nontherapeutic, non-immunogenic stabilizers and the like. Pharmaceutically acceptable carriers include any and all suitable solvents, dispersion media, coatings, antibacterial and antifungal agents, absorption delaying agents, and the like that are physiologically compatible with a compound of the present disclosure. Examples of suitable aqueous and nonaqueous carriers which may be employed in the pharmaceutical compositions of the present disclosure include water, saline, phosphate buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. The composition, if desired, can also contain a minor amount of a wetting or emulsifying agent, or a pH buffering agent.

Pharmaceutical compositions of the present disclosure may also comprise pharmaceutically acceptable antioxidants for instance (1) water-soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. Pharmaceutical compositions of the present disclosure may also comprise isotonicity agents, such as sugars, polyalcohols, such as mannitol, sorbitol, glycerol, or sodium chloride in the compositions. Prolonged absorption of the compositions may be brought about by including in the composition an agent that delays absorption, for example, monostearate salts and gelatin.

The compositions of the disclosure can be formulated as neutral or salt forms. Pharmaceutically acceptable salts include, but are not limited to those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

A variety of delivery systems and administration routes for providing antigen and/or immune modulatory agent(s) are available. Delivery systems that can be used to administer or in delivery of an antigen and/or an agent include, but are not limited to, encapsulation in liposomes, micelles, nanoparticles, microparticles, microcapsules, or other suitable carriers. Further, the molecules and/or compositions utilized in accordance with disclosed embodiments can be delivered in a controlled-release or sustained-release system. Techniques known in the art to produce sustained-release formulations comprising one or more of the subject molecules may be utilized.

In some embodiments, polymeric materials can be used to achieve delivery and/or controlled-release of the molecules. Examples of suitable polymers include, but are not limited to, poly(2-hydroxyethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), polyorthoesters, chitosan, and hyaluronic acid. A controlled-release system can be implanted, injected, or placed in proximity of or within the therapeutic target (e.g., lymph node).

In one embodiment, the compounds may be formulated to ensure proper distribution and efficacy in vivo. Pharmaceutically acceptable carriers for parenteral administration include sterile aqueous solutions or dispersions and sterile powders for the extemporaneous preparation of sterile injectable solutions or dispersion. Sterile solutions may be prepared by incorporating the active compound in the required amount in an appropriate solvent with one or a combination of ingredients, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

### Dosage and Administration

Dosage regimens in the above methods of treatment and uses are adjusted to provide the optimum desired response (e.g., a therapeutic response). For example, a single bolus (e.g., injected into a single lymph node of a subject) may be administered, or several divided doses may be administered (e.g., via injection into two or more lymph nodes of a subject). Pharmaceutical compositions may be formulated in dosage unit form for ease of administration and uniformity of dosage. Dosage unit form as used herein refers to physically discrete units suited as unitary dosages for the subjects to be treated; each unit contains a predetermined quantity of active compound or ingredient calculated to produce the desired therapeutic effect in association with any required pharmaceutical carrier. The specification for the dosage unit forms of the present disclosure are dictated by and dependent on (a) the characteristics of the active compound and the particular therapeutic effect to be achieved, and (b) any limitations in the art of compounding such an active compound for the treatment of sensitivity in individuals.

A physician skilled in the art may readily determine and prescribe the therapeutically effective amount of the ingredients and/or pharmaceutical composition required for a particular subject. The selected and effective dosage level will depend upon a variety of pharmacokinetic factors including the activity of the particular compositions of the present disclosure employed, the route of administration, the rate of excretion or release of the particular compound being employed, the duration of the treatment, other drugs, compounds and/or materials used in combination with the particular compositions employed, the age, sex, weight, condition, general health and prior medical history of the patient being treated, and like factors well known to those skilled in the medical arts. It will be appreciated that the effective dosage of the subject molecules used for treatment may increase or decrease over the course of a particular treatment. For example, the calculated dosage for administration may be based on the patient's body weight at baseline. Alternatively, a fixed dosage of the subject molecules used for treatment may be administered to a subject regardless of patient body weight.

The pharmaceutical compositions of the present disclosure may be administered by any suitable route and mode. Preferably, the disclosed compositions or compounds are administered via direct injection into one or more lymph node(s) of a subject. In some embodiments, the introduction of an antigen and immune modulatory agent into the lymph node(s) of a subject is provided for the treatment, prophylaxis, and amelioration of one or more symptoms associated with an autoimmune disease or condition, particularly T1D and graft rejection, by introducing directly into a lymph node(s) of the subject an effective amount of such a combination, or pharmaceutical composition(s) comprising the same. In some embodiments, the antigen and immune modulatory agent are introduced together directly into a single lymph node of the subject. In other embodiments, the antigen and immune modulatory agent are introduced together directly into two or more lymph nodes of the subject (e.g., with half or a portion of the total dosage introduced into one lymph node and other half or portion of the total dosage introduced into another lymph node of the subject). In other embodiments, the antigen is introduced into one or more lymph node(s), and the immune modulatory agent is introduced into another one or more lymph node(s) different from the lymph node(s) into which the antigen is introduced.

The therapeutically effective agents may be formulated for administration concurrently (e.g., wherein formulations containing the molecules are administered at the same time or within about 24 hours of each other) or sequentially (e.g., wherein a first formulation containing the antigen or immune modulatory agent is administered and, at a later time such as at least 24 hours or more after administration of the first formulation a second formulation containing the other of the antigen or immune modulatory agent is administered to the subject). As used herein, the term “combination” refers to the use of more than one ingredient or compound. The use of the term “combination” does not restrict the order in which the ingredients or compounds are administered to a subject.

Treatment of a subject with a therapeutically or prophylactically effective amount of an antigen and immune modulatory agent preferably includes a single treatment, but can also include a series of treatments. For example, a subject may be treated with formulations containing the antigen and immune modulatory agent once, or one a week, twice a week, once every two weeks, once every three weeks, once every four weeks, once every six weeks, once every two months, once a year, etc.

### Diagnostic Assays and Kits

The disclosed methods of compositions may be used to identify the presence or absence of target cells, receptors or other antigens, or the level thereof, which are circulating in blood and/or present in tissue(s) of a subject. Such antigen may be an intact antigen, or a fragment thereof that retains the ability to be detected according to methods known in the art. For example, such detection may be affected by FACS analysis using standard methods commonly used in the art. In some embodiments, a molecule coupled to or bearing a detectable label is utilized, e.g., in methods for aiding diagnosis. Examples of labels that may be used include but are not limited to a radioactive agent, an enzyme, or a fluorophore. Techniques for detection of a labeled molecule are well known in the art and include enzyme linked immunosorbent assays (ELISAs), immunoprecipitations, immunofluorescence, enzyme immunoassay (EIA), radioimmunoassay (RIA), and Western blot analysis.

As used herein, the term “labeled,” with regard to a molecule or structure, is intended to encompass direct labeling of such molecule or structure by coupling thereto (e.g., physically linking) a detectable substance (e.g., a radioactive agent or a fluorophore), as well as indirect labeling of the molecule or structure by reactivity with a detectable substance. As used herein, methods for “aiding diagnosis” refers to methods that assist in making a clinical determination regarding the classification, or nature, of a disease or condition, and may or may not be conclusive with respect to the definitive diagnosis.

A biological sample may be obtained to assess an immune response of a subject. The term “biological sample” encompasses a variety of sample types obtained from a subject and can be used in a diagnostic or monitoring assay. The definition encompasses saliva, blood, and other liquid samples of biological origin, solid tissue samples such as a biopsy specimen or tissue cultures or cells derived therefrom, and the progeny thereof, for example, cells obtained from a tissue sample collected from a subject diagnosed with or exhibiting symptoms of a disease or condition (e.g., an autoimmune disease or condition). The definition also includes samples that have been manipulated in any way after their procurement, such as by treatment with reagents, solubilization, or enrichment for certain components, such as proteins or polynucleotides, or embedding in a semi-solid or solid matrix for sectioning purposes.

The present disclosure also encompasses kits for prophylaxis and/or treatment of an autoimmune disease or condition. The kit can comprise an antigen and an immune modulatory agent in a single formulation, or in separate formulations for mixing together. The kit can comprise a biodegradable material for use in preparing a formulation for direct intra-LN administration. The kit can comprise a suitable buffer and pharmaceutically acceptable excipients, carriers, and the like. The components or ingredients can be included in separate containers, or a single container with separate compartments. The containers can include sterile components, vials, ampules, tubes, a bolus, etc. The kit may comprise a needle suitable for direct administration of a composition of this disclosure into one or more lymph nodes, and a syringe, and/or other components for use in lymph node injection. The kit can additionally comprise printed material, such as instructions, and/or an indication that the kit is for use in treating, for example, a subject diagnosed with or exhibiting symptoms of an autoimmune disease or condition, and in particular T1D or graft transplant rejection.

### Intra-LN Vaccine Deposition and Treatment Strategies

As discussed above, LNs are key tissues that integrate immune signals to coordinate adaptive immune responses, and thus are important targets for vaccines. Directly controlling the signals and local environment in LNs enables potent and safe immunotherapies without cell isolation, engineering, and reinfusion. In accordance with disclosed embodiments, intra-LN injection of immune signal-loaded biomaterial depots is utilized to directly control vaccine deposition.

Various embodiments of the disclosure are illustrated using PLGA as a representative biodegradable depot or carrier material. However, various other materials, including but not necessarily limited to biocompatible and/or biodegradable polymers, may be utilized in accordance with the present disclosure, provided that the selected antigen and/or the immune modulatory agent can be exposed to immune cells in the lymph nodes concurrent with or subsequent to administration. For example, as an alternative to PLGA, the biodegradable material can comprise poly(glycolide) (PGA), poly(L-lactide) (PLA), or poly(beta-amino esters). In some embodiments, the biodegradable material may be a hydrogel, an alginate, or a collagen. In some embodiments, the biodegradable material can comprise a polyester, a polyamide, or polyethylene glycol (PEG). The antigen(s) and/or agent(s) may be coupled to or encapsulated by the biodegradable polymer carrier. In some embodiments, the antigen(s) and/or the agent(s) are released over a period of time, such as in an extended release formulation where the components are released on the time scale of hours, days, or weeks. The carriers or depots are preferably microparticles having a size sufficiently large such that diffusion from LN(s) is limited or restricted, e.g., having a diameter of between about 1-100 μm, preferably between about 1-10 μm, more preferably between about 2-5 μm.

While soluble signals quickly drain from LNs, the present disclosure provides for the synthesize and introduction of carriers (e.g., microparticles (MPs)) that are too large to drain out of LNs and thus are diffusion-limited (Jewell, C. M. et al. (2011) In situ engineering of the lymph node microenvironment via intranodal injection of adjuvant-releasing polymer particles. Proc. Natl. Acad. Sci. 108, 15745-15750). Instead, the MPs slowly degrade, locally concentrating immune cues that condition the local LN environment to direct immune cell fate in LNs. Application of this precision approach to T1D and islet transplantation localizes tolerizing signals in LNs for tunable times and concentrations, obviating the need for systemic administration of high doses currently utilized in an effort to restrain T1D and during islet transplantation. This platform also allows for the direct evaluation of how immune cues impact the local LN microenvironment during antigen-specific autoimmunity in treated and untreated nodes.

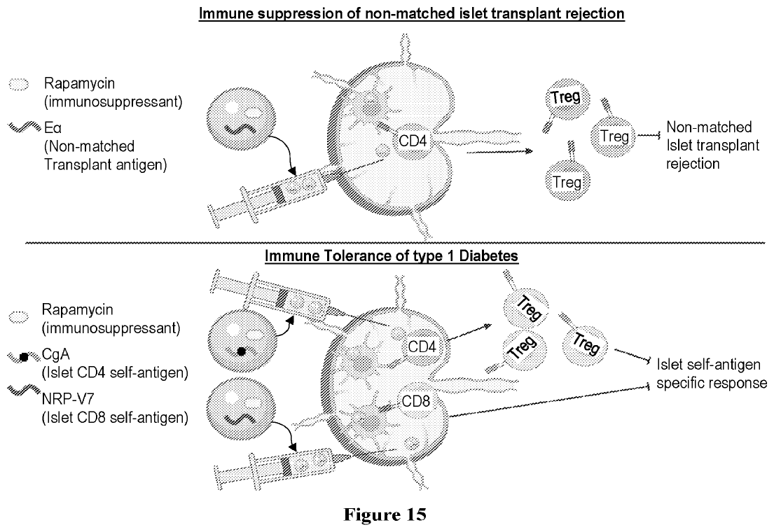

Here we have applied this system to two distinct T1D contexts: i) pre-clinical models of CD4 and CD8-mediated T1D; and ii) an allogenic islet transplant model in which non-matched islets are transferred to recipients after islet depletion (FIG. 15). We synthesized microparticles (MPs) co-encapsulating rapamycin and three distinct T1D self-antigens targeted in T1D or graft rejection after islet transplantation. In all three models, MP formulations with appropriate self-antigens effectively prevented disease, and co-encapsulation of self-antigen with rapamycin was beneficial in providing optimal protection in each case. MP treatment promoted expansion of antigen-specific T_{REG}, in treated and untreated LNs, and resulted in structural reorganization of LNs with microdomains associated with induction of tolerance. Finally, antigen-specific T_{REG} displayed markers of enhanced persistence, and a single MP treatment stopped disease even when disease induction was delayed relative to treatment.

Thus, as demonstrated herein, the methods, compositions, and systems of the present disclosure are effective for use in treating T1D and islet transplantation rejection. In both of these applications, the direct delivery of the target antigen and immunomodulatory cues to lymph nodes (LNs) provided potent efficacy against disease through the induction of antigen-specific regulatory T cells (Treg) and increased tolerance-promoting regions in lymph nodes. Additionally, the disclosed methods and platforms promoted the formation of persistent, memory-like Tregs, which provide long-lasting protection following a single treatment. Thus, the disclosed methods, compositions, and systems may be utilized to provide tolerance-based protection against various autoimmune diseases and conditions, including T1D and graft transplant rejection.

Additional characteristics and features of the present disclosure will be further understood through reference to the following additional examples and discussion, which are provided by way of further illustration and are not intended to be limiting of the present disclosure.

A library of immunosuppressive microparticles (MPs) co-encapsulating rapamycin, a small molecule mTOR inhibitor, and autoantigens associated with or relevant to T1D (e.g., CgA, NRP-V7) and allograft rejection (Eα) were synthesized by double emulsion. In in vitro co-cultures with dendritic cells and transgenic T cells displaying T cell receptors specific for encapsulated antigens, MP treatment reduced T cell proliferation, inhibited secretion of IL-17 and IFNγ and increased regulatory T cell (T_{REG}) differentiation. Additionally, MP treatment increased expression of markers associated with cell persistence among T_{REG}. Direct lymph node (LN) injection of tolerogenic MPs encapsulating relevant antigens enhanced survival in models of T1D and islet transplantation. Co-encapsulation of antigen and rapamycin was provided for maximum efficacy in both models. After LN injection of non-obese diabetic (NOD) mice with tolerogenic MPs, antigen specific T cells are primed early in treated LNs with low mTOR activity. At later timepoints, antigen specific T cells are highly expanded and activated in distant LNs and are polarized toward T_{REG}. Following LN injection, antigen is presented in MHC-II in treated and distant LNs. Antigen presentation in distant LNs is not inhibited after administration of FTY720, which blocks lymphocyte egress from LNs. Microparticle treatment promoted microdomains in LNs previously reported to be associated with tolerance, characterized by increases in laminin α4:α5 ratios and antigen specific T_{REG} accumulation in high endothelial venules and cortical ridges. Finally, local LN remodeling correlates with increased antigen specific T_{REG} and reduced T cell activation in grafts.

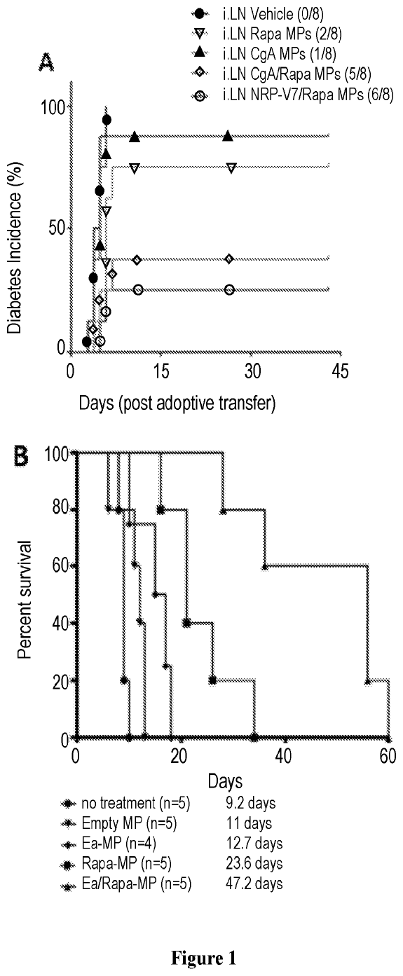

As demonstrated herein, intra-LN depots protect against onset of T1D and prolong allograft survival in mouse models. To test the efficacy of this platform in the context of T1D, pancreatic islet antigen (CgA or NRP-V7) was encapsulated in polymer microparticles (MPs) alongside the regulatory drug rapamycin. MPs were delivered via intra-LN injection into non-obese diabetic (NOD) mice, followed by adoptive transfer of activated islet-specific T cells to induce disease. Mice treated with MPs containing both antigen and rapamycin were protected from T1D onset compared to the vehicle control (FIG. 1a). To test MP efficacy in a transplant model, naïve mice were treated via intra-LN injection with MPs containing alloantigen (Eα) and rapamycin, followed by transplant of donor pancreatic islet cells. Islet cell transplant survival was prolonged in mice receiving MPs containing both Eα and rapamycin, compared with control groups (FIG. 1b). Taken together, these data demonstrate that the disclosed platform and methodologies provide protection against both T1D onset and prolonged transplant survival in mouse models.

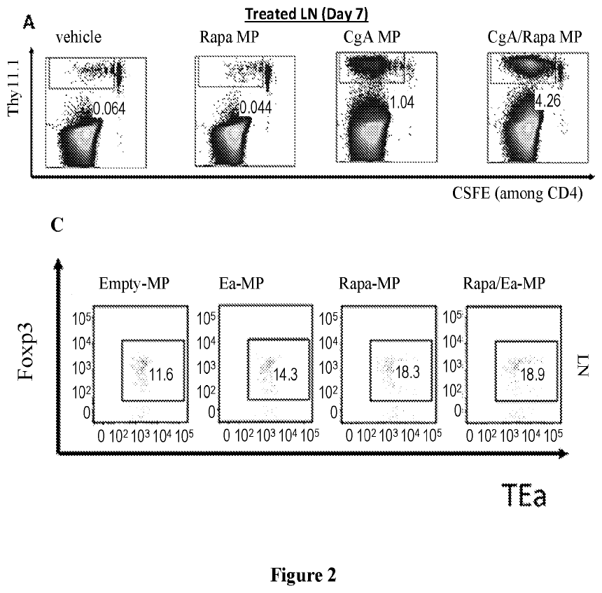

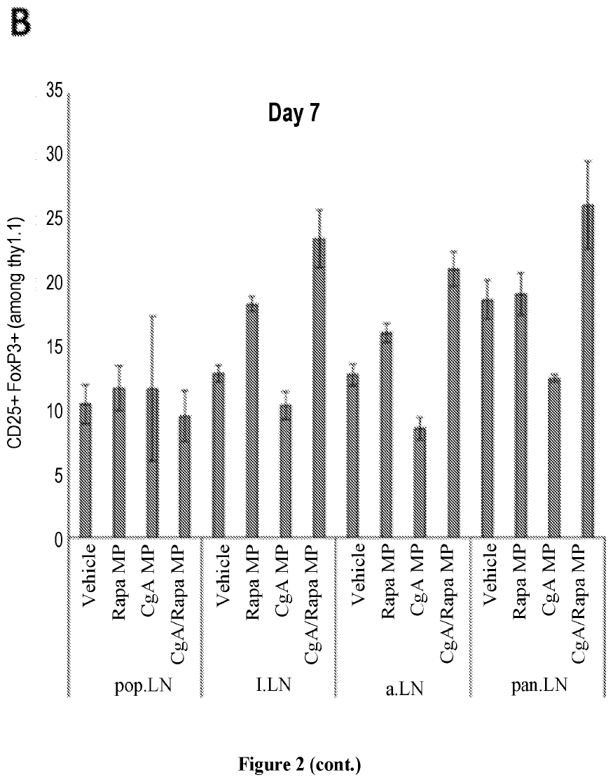

Intra-LN treatment with antigen-rapamycin MPs promotes expansion of antigen-specific T_{REGS}. To understand the mechanism underlying the efficacy of this technology, we examined the phenotype of antigen-specific T cells in both T1D and transplantation models. For T1D, NOD mice were treated via intra-LN injection with CgA-rapamycin MPs, followed by adoptive transfer of labeled antigen-specific T cells. Seven days post-transfer, the phenotype of antigen-specific cells from LNs was examined. We found that treatment with CgA-rapamycin MPs led to increased proliferation of antigen-specific T cells, and increased the proportion of T_{REGS}, as measured by dual expression of CD25+ and FoxP3+ markers (FIG. 2a-b). Similarly, we observed an increase in the proportion of T_{REGS} in mice treated with rapamycin/Eα-MP compared to antigen-only or empty MP treatments (FIG. 2c). These studies demonstrate that intra-LN delivery of MPs induces antigen-specific T_{REGS} that likely play a tolerogenic role in mouse models.

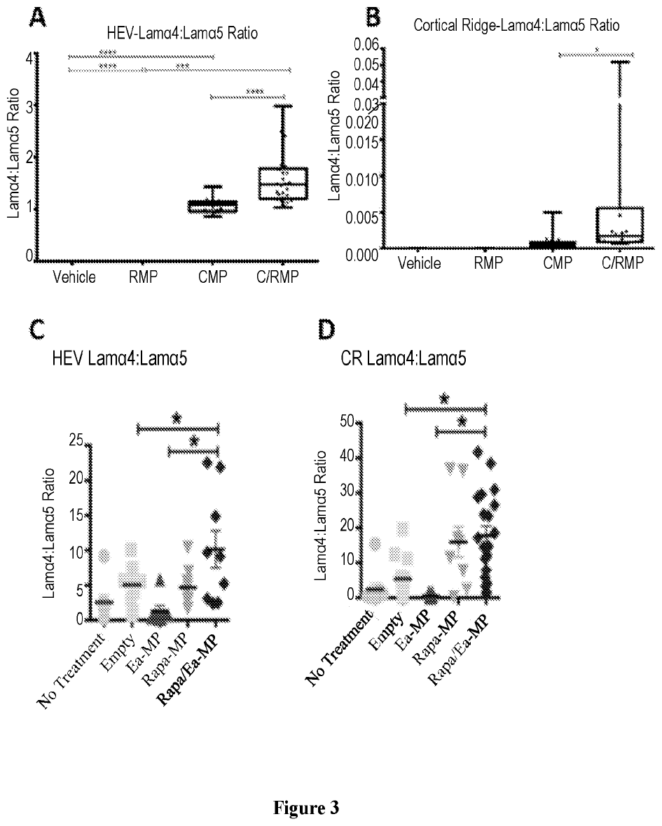

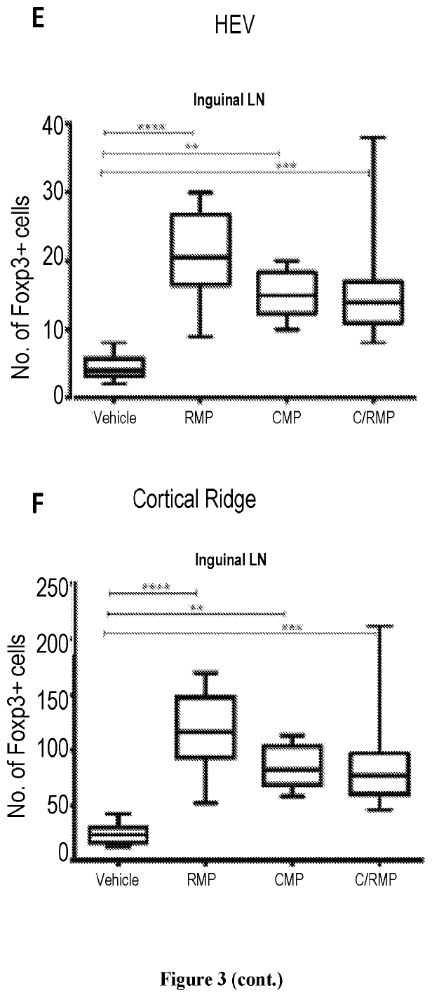

Further, intra-LN MPs stimulated an increase in tolerance-promoting LN regions. In addition to the regulatory phenotype adopted by antigen-specific cells, we studied how intra-LN delivery MPs impacts the structure of the LNs by examining changes in the ratio of lamα4:lamα5 extracellular matrix protein expression both in the high endothelial venules (HEV) and cortical ridge (CR), an indicator of a more tolerogenic immune microenvironment. For T1D, NOD mice were treated via intra-LN injection with CgA-rapamycin MPs. LNs were then harvested and stained for lamα4 and lamα5. MPs containing both antigen and rapamycin promoted an increase in the lamα4:lamα5 ratio compared to control groups (FIG. 3a-b). In our transplantation model, treatment with Eα/rapamycin MPs also led to an increased lamα4:lamα5 ratio compared to other treatment groups (FIG. 3c-d), indicating that structural changes following MP delivery are suggestive of tolerance accompany disease prevention and transplant survival (see FIG. 1).

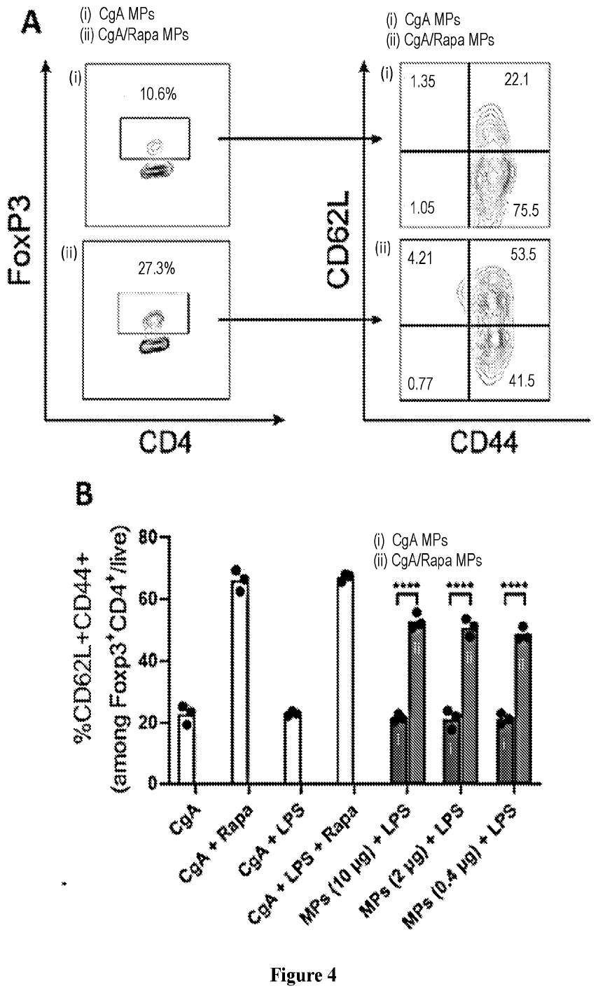

As also demonstrated herein, antigen-rapamycin MPs promote a memory-like T_{REG} phenotype in vitro. Memory T_{REGS} provide long-lasting protection from disease recurrence or reactivity to transplants. To test whether T_{REGS} induced by MP treatment exhibit a memory-like phenotype, mouse CD11c+ dendritic cells were isolated and treated with CgA-containing MPs with or without rapamycin. The following day, transgenic Bdc2.5 T cells with a CgA antigen specificity were added to the plate. After 3 days, T cells were stained for FoxP3 and CD25 to examine T_{REG} polarization, and further stained with CD44 and CD62L, preliminary markers of memory cell formation. Compared to antigen and control MPs, MPs containing both antigen and rapamycin increased the proportion of CD62L+CD44+ double-positive cells among T_{REGS} (FIG. 4a-b), indicating that treatment with tolerogenic MPs provides long-term protection.

Thus, intra-LN delivery of MPs promotes antigen-specific tolerance to indications of T1D and transplantation. In exemplary models disclosed herein, utilization of the methods, compositions, and systems of the present disclosure are efficacious in treating and/or preventing disease and promoting survival. Accompanying this efficacy are changes in LN structure as well as the phenotype and proliferation of antigen-specific T cell populations that indicate both tolerance and memory toward delivered antigen.

### Materials and Methods

Cell and Animals. NOD (NOD/shiltJ), BDC2.5 (NOD.Cg-Tg(TcraBDC2.5,TcrbBDC2.5)1Doi/DoiJ), NRP-V7 (NOD.Cg-Tg(TcraTcrbNY8.3)1Pesa/DvsJ), Tea (B6.Cg-Tg(Tcra,Tcrb)3Ayr/J) C57B,L/6J and BalbC mice were purchased from The Jackson Laboratory. BDC2.5-thy1.1 mice were obtained from the Genetically Modified NOD Mouse Core Facility, Harvard Medical School, Pathology, NRB 1052G, 77 Avenue Louis Pasteur, Boston, MA 02115.

Microparticle Synthesis and Characterization. Degradable PLGA (Sigma Aldrich) MPs were synthesized as previously described via double-emulsion. Briefly, an initial emulsion was generated by sonicating 80 mg PLGA with 4 mg rapamycin (LC labs) dissolved in 5 mL dichloromethane with 500 μL of water containing 1 mg of antigen. This initial emulsion was then homogenized with 40 mL water containing 1% polyvinyl alcohol (Sigma) at 16,000×g for 3 min, and was allowed to evaporate overnight with stirring to allow evaporation of dichloromethane. MPs were then filtered through a 40 μm strainer and collected via centrifugation at 5000×g for 5 min at 4° C. Supernatants were removed, and MPs were washed three times with 1 mL water. MPs were then resuspended in 1 mL water for in vitro and in vivo studies. Particle size was determined using an LA-950 laser diffraction analyzer (Horiba). To determine rapamycin loading, a known mass of MPs was dissolved in dimethyl-sulfoxide, and the absorbance at 278 nm was determined using UV/VIS spectrophotometry. Absorbance values were fit to a standard curve of known rapamycin concentrations to calculate rapamycin loading per mass of MPs. To determine peptide loading, Micro BCA Protein Assay Kit (Thermofisher Pierce) was used as previously described. For preparation of fluorescently labeled MPs, 5 μL of DiR was dissolved with PLGA prior to the primary emulsion during MP synthesis, and FITC-labeled CgA was used in place of unlabeled CgA during synthesis.

Flow cytometry. The following antibodies were used for flow cytometry: CD4 (clone GK1.5), Thy1.1 (clone OX7), CD8 (clone 53-6.7), CD62L (clone MEL-14), CD44 (clone IM-7), CD127 (clone SB/199), BCL-2 (clone 3F11), foxp3 (clone MF23), CD69 (clone H1.2F3), anti-s6 pS235/S236 (clone N7-584), IFNγ (clone XMG1.2), IL-17 (clone TC11-18H10). All antibodies for flow cytometry were purchased from BD biosciences. Mice were euthanized and lymph nodes collected and processed into single cell suspension by mechanical dissociation through a 40 μm cell strainer. Cells were washed once with PBS containing 1% w/v BSA (FACS buffer) and stained with anti CD32/CD16 (BD, Clone 2.4G2) for 10 min at room temperature. Cells were then stained with surface marker antibodies for 20 minutes at room temperature. Cells were then washed two times with FACS buffer and were either analyzed immediately or stained for intracellular markers. For intracellular stains, cells were fixed and permeabilized by incubating with fix/perm buffer from the Foxp3/Transcription factor staining buffer set (eBioscience) for 40 min at 4 degrees C. Cells were stained with antibodies against intracellular markers for 30 min at 4 degrees C., followed by two washes with perm/wash buffer. Cells were analyzed with a Canto II (BD) or FACSCelesta (BD). DAPI or LIVE/DEAD Fixable Viability Dye (Thermofisher) were used for cell viability.

In vitro T cell co-cultures. CD11c dendritic cells were isolated from mouse splenocytes using positive magnetic selection (Miltenyi) and were plated at 100,000 cells/well in 96 well plates. For co-cultures utilizing BDC2.5 or NRP-V7 T cells, dendritic cells (DCs) were isolated from NOD mice. For co-cultures utilizing Eα T cells, DCs were isolated from C57-BL6J mice. Following isolation, DCs were stimulated with lipopolysaccharide (LPS) and treated with indicated doses of MPs for 24 hrs. T cells were then isolated from splenocytes using negative magnetic selection (Stemcell), labeled with CFSE or CellTrace Violet (Thermofisher), and 300,000 T cells were co-cultured with MP treated DCs for 3 days. CD4⁺ selection was used for BDC2.5 and Eα T cells, and CD8⁺ selection was used for NRP-V7 T cells. After 3 days, co-cultures were stimulated with a cell stimulation cocktail (Thermofisher) in the presence of BFA (Biolegend) for 4 hours, followed by surface and intracellular marker antibody staining and analysis flow cytometry to assess proliferation and determine T cell phenotype.

Peptides. CgA (YVRPLWVRME), Eα (ASFEAQGALANIAVDKA) and NRP-V7 (KYNKANVFL) were synthesized by Genscript with purity >98%.

Intra-LN injection. Intra-LN injection of mice was performed as previously described. Briefly, hair surrounding the injection site was removed using a mild depilatory cream, and mice were injected subcutaneously at the tail base with a tracer dye (Evan's Blue). 1 day after tracer dye injection, both inguinal LNs were identified and directly injected with 1 mg of MPs suspended in sterile water using a 31 g syringe.

Diabetes induction through NRP-V7 T cell adoptive transfer. 8 week old female NOD mice were immunized intra-LN with indicated MP treatments. Splenocytes from NRP-V7 mice were stimulated with 1 μM NRP-V7 peptide at 2×10⁶ cells/mL in complete media for 3 days, washed with PBS, and injected i.v. into host NOD mice at 15×10⁶ cells/mouse. Blood glucose was monitored daily following T cell infusion, and mice were considered diabetic at a blood glucose level >250 mg/dL.

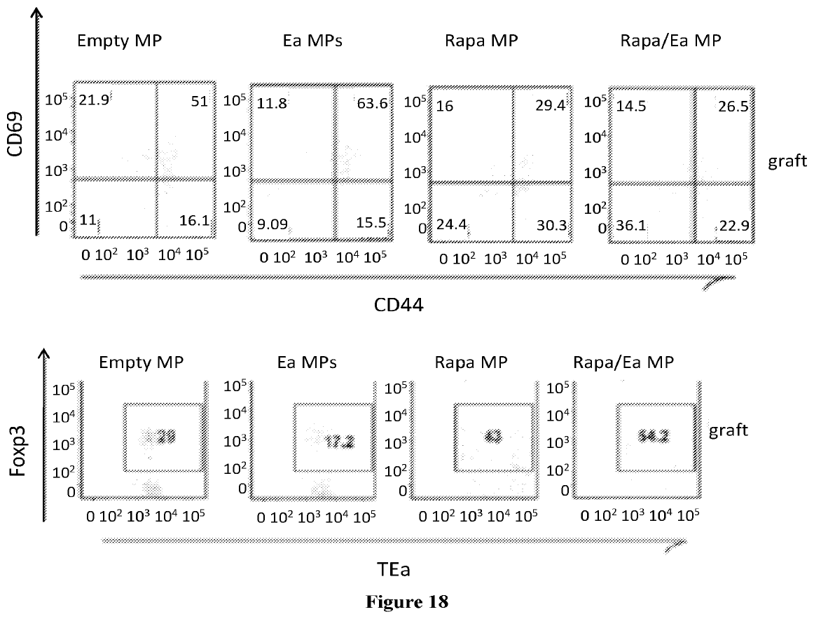

Islet transplantation studies. 8-10 week old C57BL/6 mice were induced with diabetes by intra-peritoneal injection of 180 mg/kg Streptozotocin. Islets were implanted and lymph nodes were injected with indicated treatments on the day host mice became diabetic. To collect allogenic islets, BALB/c mice were euthanized and the common bile duct was injected with 3 mL cold Hanks' buffer containing 1.5 mg/mL of collagenase-P (Roche Diagnostics, Indianapolis, IN). Pancreata was surgically excised and digested at 37° C. for 15 min. The digested pancreas was disrupted by vigorous shaking, and the suspension was washed twice with RPMI 1640 containing 10% fetal bovine serum. Pancreatic islet separation was performed by centrifugation on a discontinuous Ficoll (Sigma) gradient of 11%, 21%, 23%, and 25%. Islets were picked from the second layer, and 400 islets were transplanted beneath the renal capsule of host mice (FIG. 18). Blood glucose was monitored daily, and mice were euthanized after two consecutive readings of blood glucose >300 mg/dL.

BDC2.5 T cell adoptive transfer studies. 8-10 week old female non-obese diabetic (NOD) mice were immunized intra-LN with indicated MP treatments. One day following intra-LN treatment, BDC2.5 T cells were isolated from splenocytes of BDC2.5 mice BDC2.5-thy1.1 mice using CD4 negative magnetic selection (Stemcell) per the manufacturer's instructions and were adoptively transferred to host NOD mice T cells/mouse. In studies using BDC2.5 T cells, cells were labeled with CFSE prior to adoptive transfer. At indicated times following T cell transfer, host mice were sacrificed and LNs were processed and analyzed by flow cytometry. BDC2.5 T cells were identified based on CFSE signal when using BDC2.5 T cells or the expression of thy1.1 when using BDC2.5-thy1.1 T cells.

In vivo imaging. 8 week old NOD mice were immunized intra-LN with DiR MPs encapsulating FITC labeled CgA. 1 and 3 days after MP treatment, mice were euthanized, indicated LNs were excised and imaged for DiR and FITC signal by IVIS.

Immunohistochemistry and histology. The following antibodies were used for immunohistochemistry: ERTR7 (clone sc-73355), CD11c (cloneHL3), Laminin alpha4 (clone 775830), laminin alpha 5 (polyclonal), Foxp3 (clone NRRF-30), Vα2 TCR (clone B20.1). For immunohistochemistry, LNs and spleens were frozen with OCT compound (Scigen Tissue-Plus, Gardena, CA). Frozen sections were cut at 6 μm, fixed with cold acetone, blocked with 5% goat or donkey serum (Jackson ImmunoResearch, West Grove, PA) and incubated with the indicated antibodies and DAPI. Samples were further processed as described previously (Li, L. et al. (2020) The lymph node stromal laminin α5 shapes alloimmunity. J. Clin. Invest. 130, 2602-2619; Simon, T. et al. (2019) Differential Regulation of T Cell Immunity and Tolerance by Stromal Laminin Expressed in the Lymph Node. Transplantation 103, 2075-2089; Warren, K. J. et al. (2014) Laminins affect T cell trafficking and allograft fate. J. Clin. Invest. 124, 2204-2218). Images were acquired with a Nikon Eclipse 700 (Nikon, Melville, NY, USA) and analyzed with Volocity image analysis software (Perkin Elmer, Waltham, MA). The positive staining area percentage was quantified based on at least three independent experiments with 3 sections per LN and 3-5 fields/section. Number of mice is indicated in figure legends.

Statistical analysis. Analyses were carried out with Graphpad Prism (version 8.4.3). The specifics of statistical tests for each experiment and the number of replicates are detailed in figure legends. Error bars in all panels represent the mean±standard deviation and p values <0.05 were considered significant with levels of significance were defined as *p<0.05, **p<0.01, ***p<0.001, ****p<0.0001.

### Results and Discussion

Tolerogenic MPs promote antigen-specific regulatory responses across multiple T1D-relevant self-antigens. We hypothesized intra-LN depots could promote antigen-specific tolerance against pancreatic antigens and transplanted islet antigens. Since in the NOD model of T1D tolerance can span multiple epitopes, we hypothesized our platform would enable different antigens to be used interchangeably to drive tolerance across key epitopes underpinning T1D pathology. To test this, we selected the NOD model of T1D—where the NRP-V7 and Chromogranin A (CgA) pancreatic self-antigens are targeted by T cells, and a non-matched islet transplant model—where the Eα alloantigen is targeted by the recipient's immune system after transplant of donor islets.

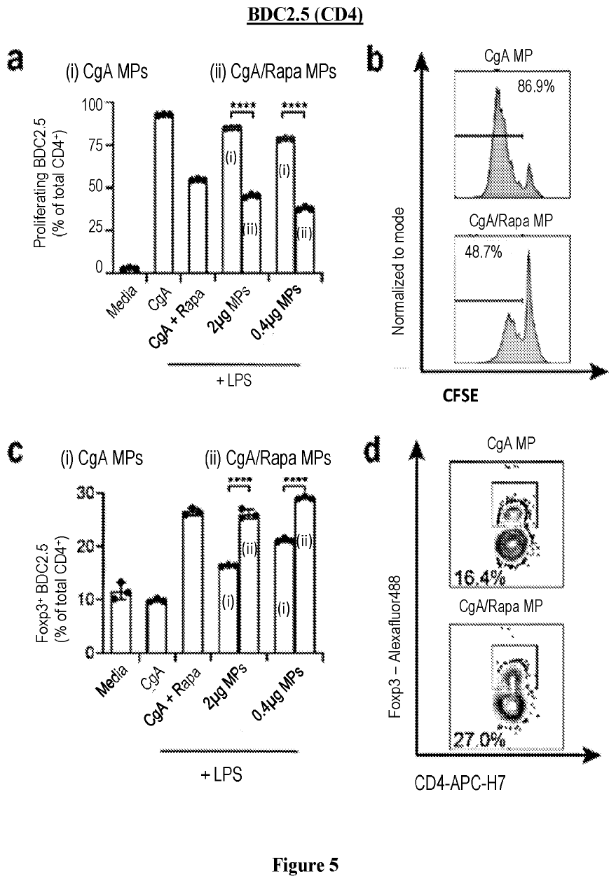

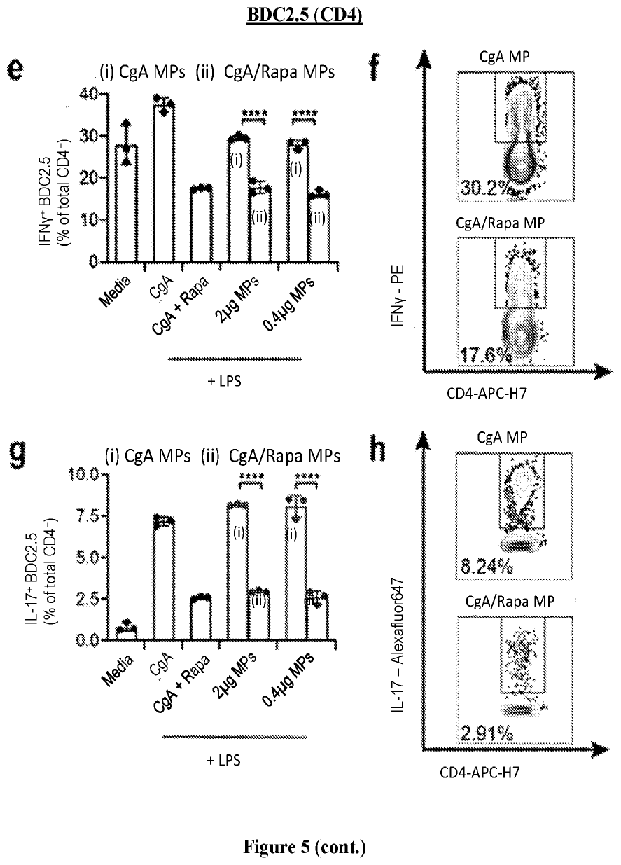

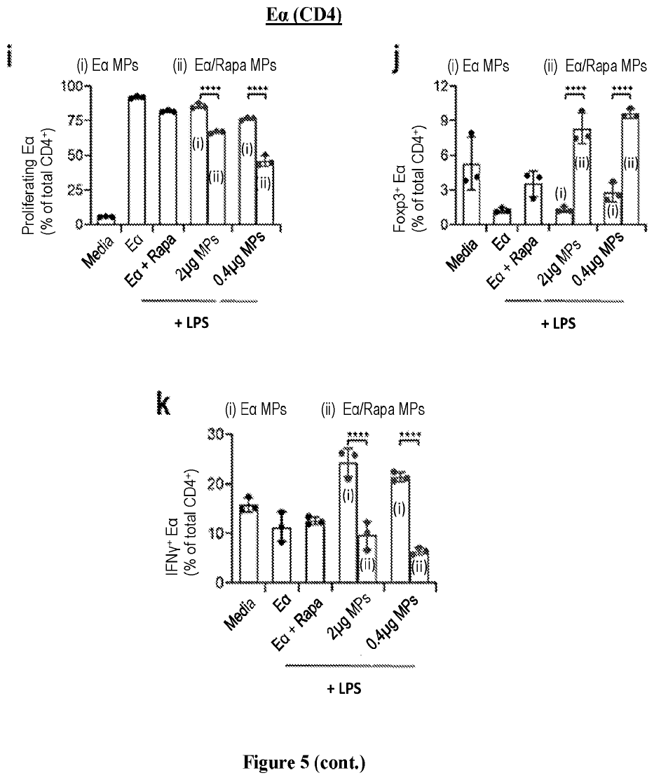

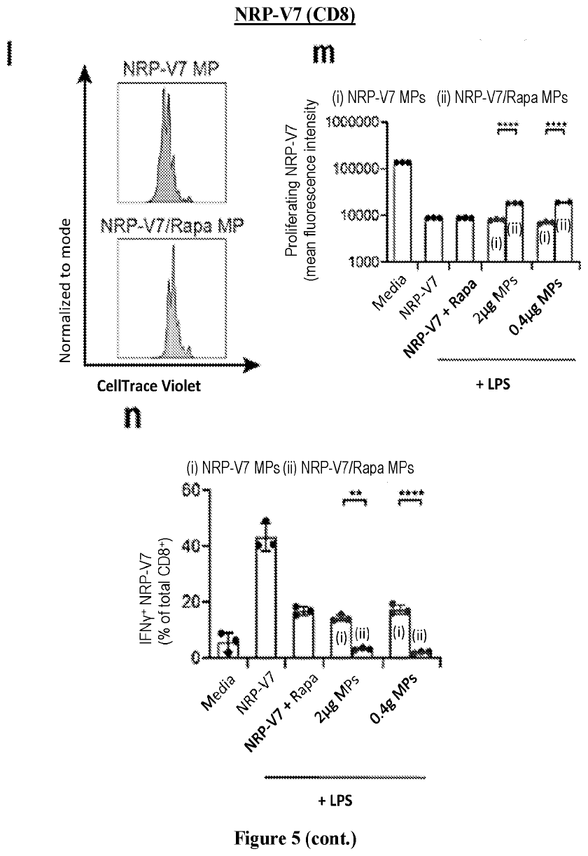

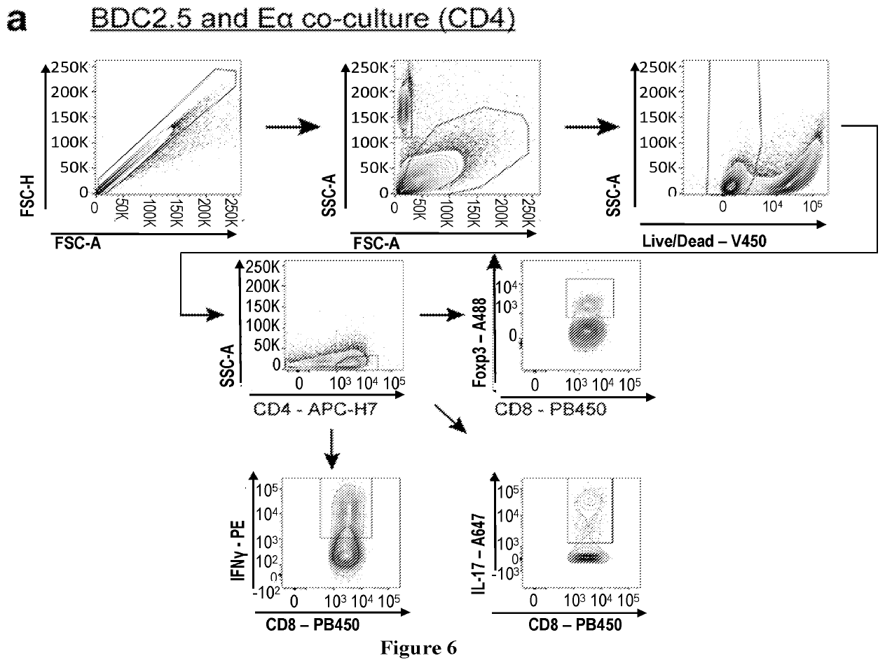

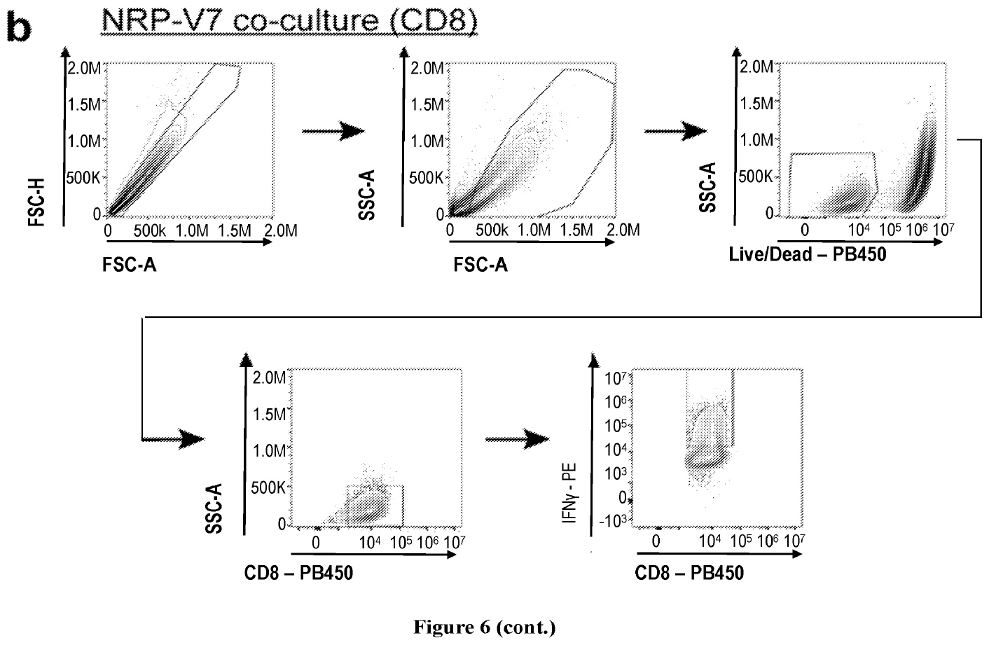

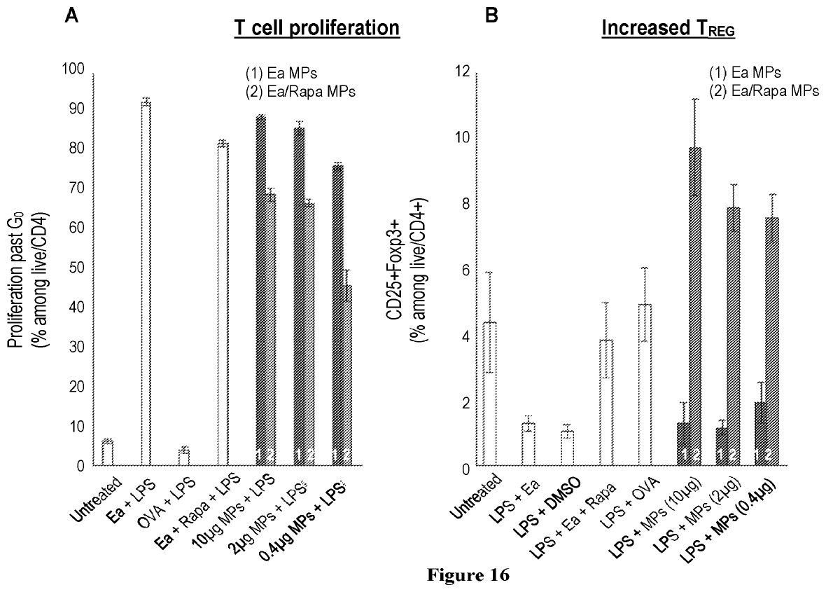

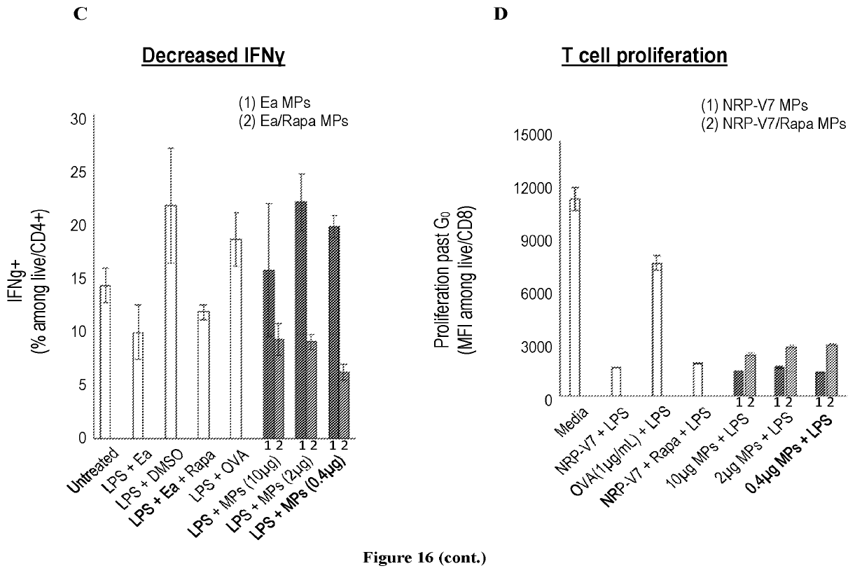

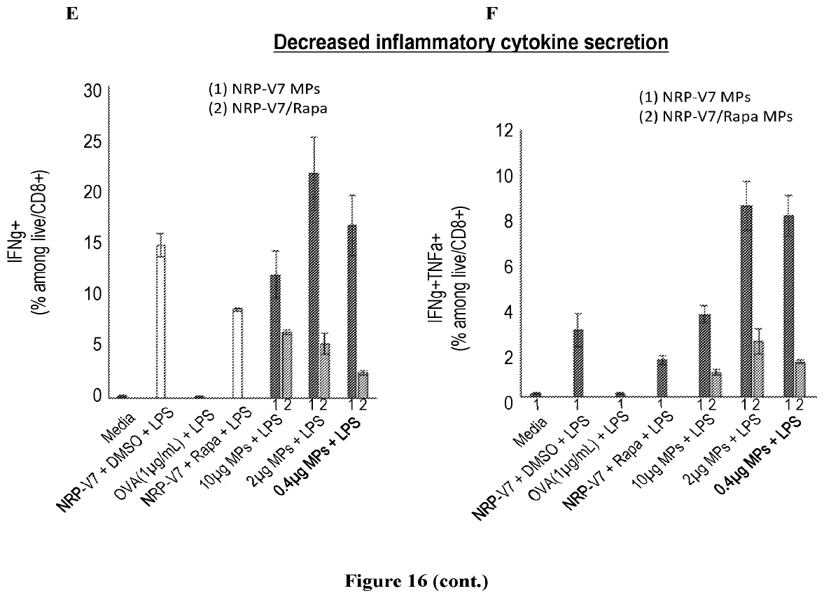

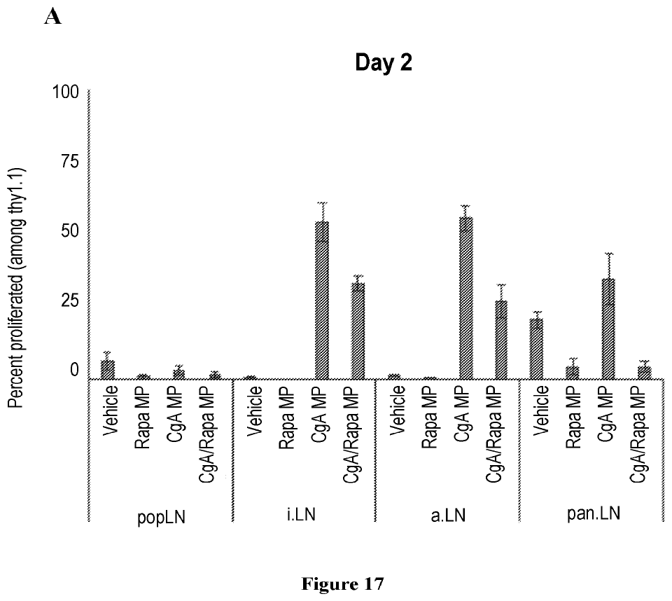

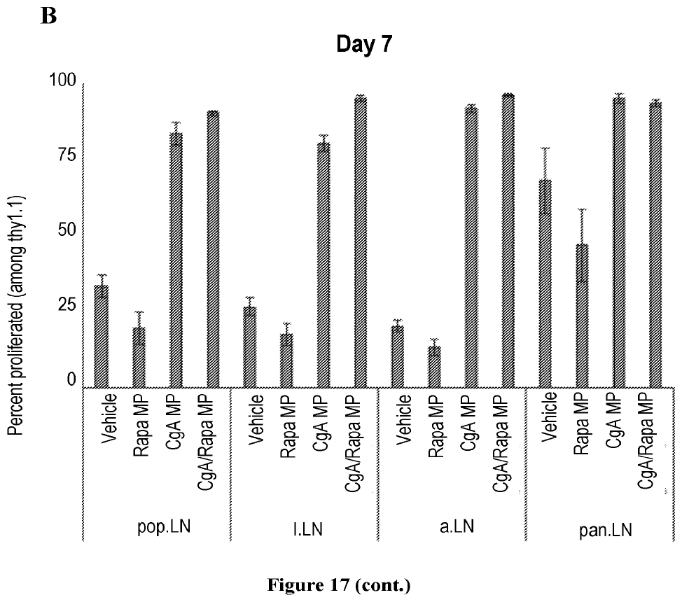

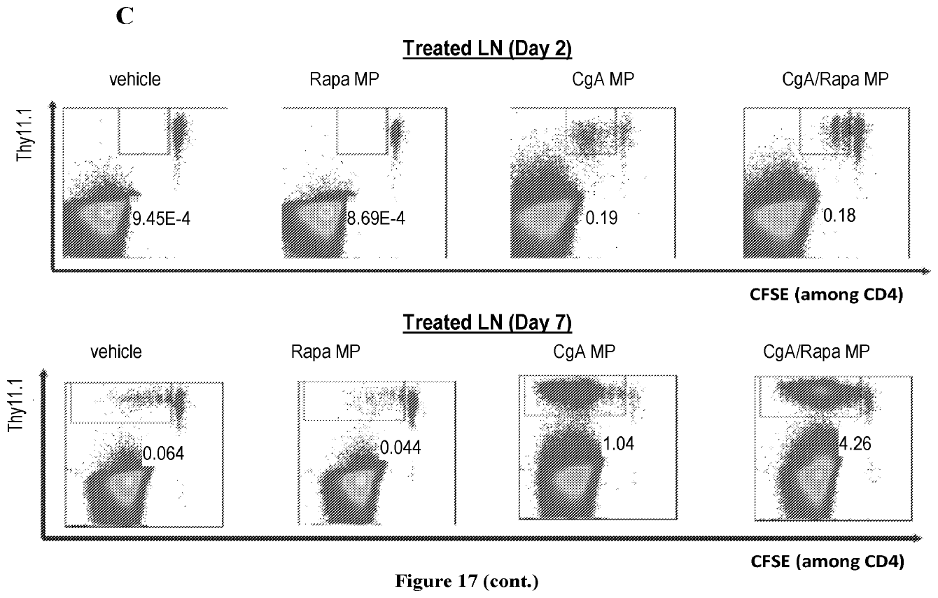

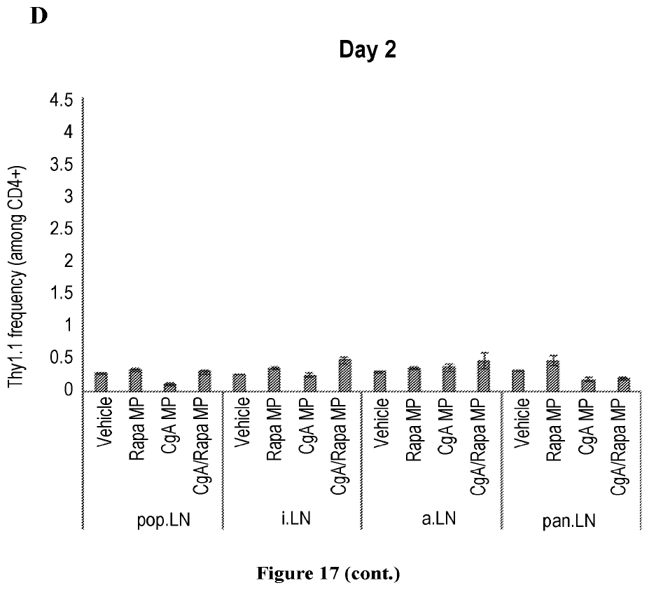

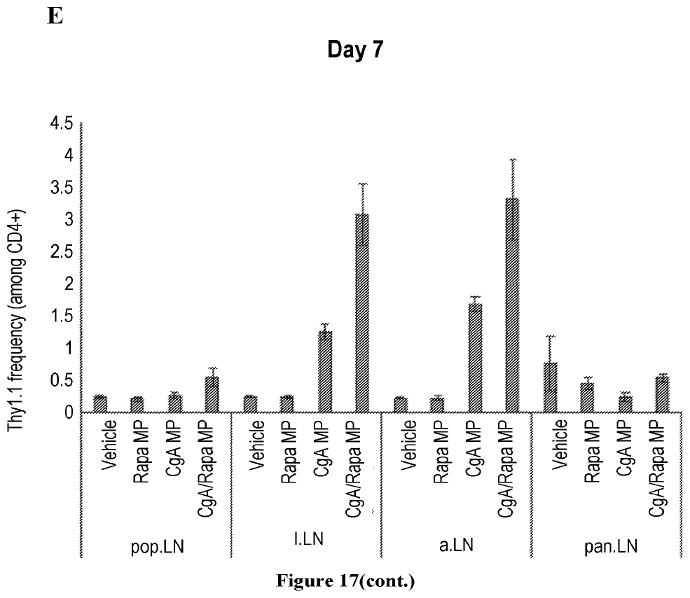

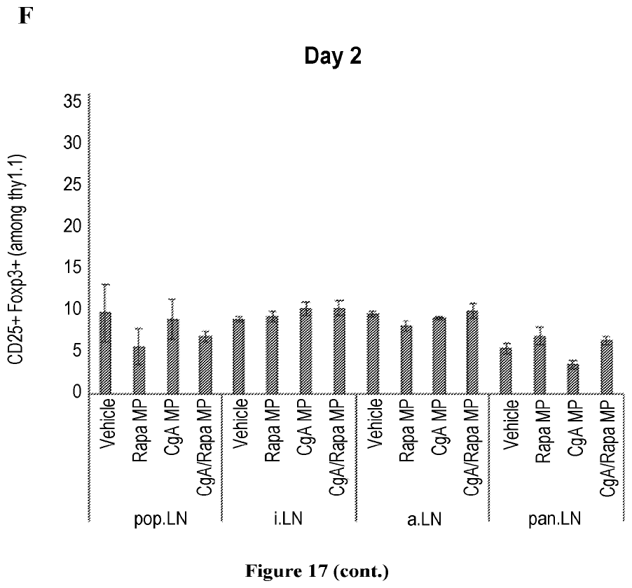

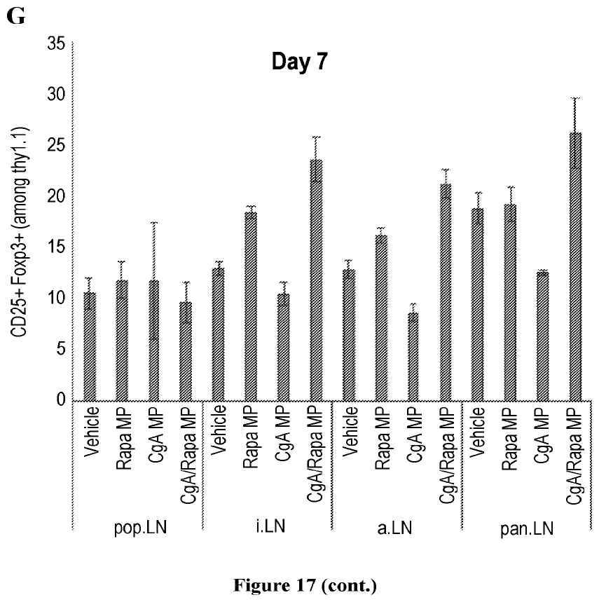

Poly(glycolide-co-lactide) (PLGA) MP depots were synthesized co-encapsulating rapamycin with each self- (e.g., NRP-V7, CgA) or alloantigen (e.g., Eα). In co-cultures consisting of DCs from NOD mice and BDC2.5 transgenic CD4 T cells—which display T cell receptors specific for CgA antigen presented by DCs, CgA/rapamycin MP partially inhibited T cell proliferation compared to CgA (FIG. 5a, 5b). However, phenotypic analysis of the expanding BDC2.5 T cells revealed CgA/rapamycin MP significantly increased T_{REG} frequency (FIG. 5c, 5d). This polarization was also associated with decreased secretion of T_{H}1- and T_{H}17-associated inflammatory-cytokines, IFNγ (FIG. 5e, 5f; FIG. 6a), and IL-17 (FIG. 5g, 5h; FIG. 6a), respectively. Analogous shifts from inflammation to tolerance (e.g., T_{REG}) were observed during co-culture of DCs with Eα-specific transgenic CD4 T cells treated with Eα/rapamycin MPs, but not Eα MPs (FIG. 5i, 5j, 5k; see also FIG. 16a, 16b, 16c). Lastly, during co-culture of DCs and NRP-V7 specific CD8 T cells, NRP-V7/rapamycin MP significantly restrained proliferation and IFNγ secretion (FIG. 5i, 5m, 5n; FIG. 6b; see also FIG. 16d, 16e, 16f). Together these data demonstrate antigen-specific tolerization and control of inflammation in three distinct T1D contexts that span self- (e.g., CgA, NRP-V7) and alloantigen (e.g., Eα), as well as CD4- (CgA, Eα) and CD8-driven (e.g., NRP-V7) inflammatory T cells.

MPs encapsulating relevant antigens and rapamycin promote suppressive T cell responses in vitro. BDC2.5 T cell co-cultures were treated with CgA MP or CgA/rapamycin MPs. (FIG. 5a) Quantification of BDC2.5 T cell proliferation and (FIG. 5b) representative flow cytometry plots of CFSE dilution are shown. (FIG. 5c) quantification of Foxp3 expression and (FIG. 5d) representative flow cytometry plots are shown. (FIG. 5e) quantification of IFNγ expression and (FIG. 5f) representative flow cytometry plots are shown. Eα T cell co-cultures were treated with Eα MP or Eα/rapamycin MPs. Quantification of (FIG. 5i) proliferation, (FIG. 5j) Foxp3 expression and (FIG. 5k) IFNγ expression are shown. NRP-V7 T cell co-cultures were treated with NRP-V7 MP or NRP-V7/rapamycin MPs. (FIG. 5l) representative flow cytometry plots showing proliferation and quantification of (m) proliferation and (FIG. 5n) IFNγ secretion are shown. N=3 individual culture wells for all experiments. Plots represent mean±s.d. and one way ANOVA with Tukey's post testing were used to compare treatment groups and comparisons between matched doses of CgA and CgA/rapamycin MPs are shown.

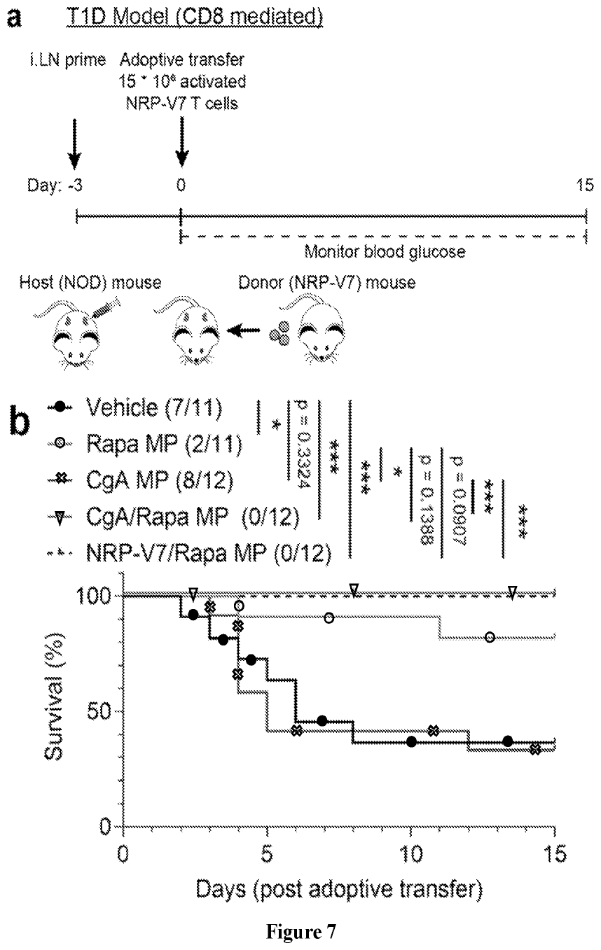

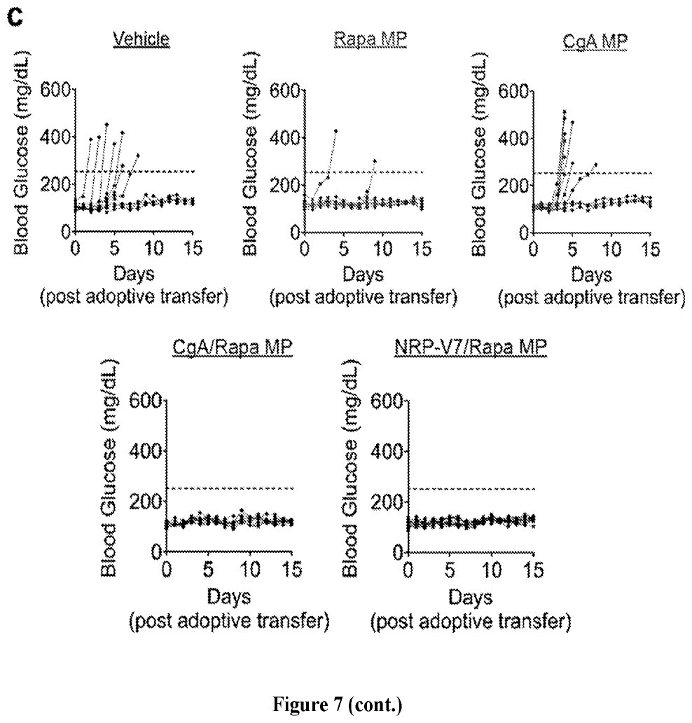

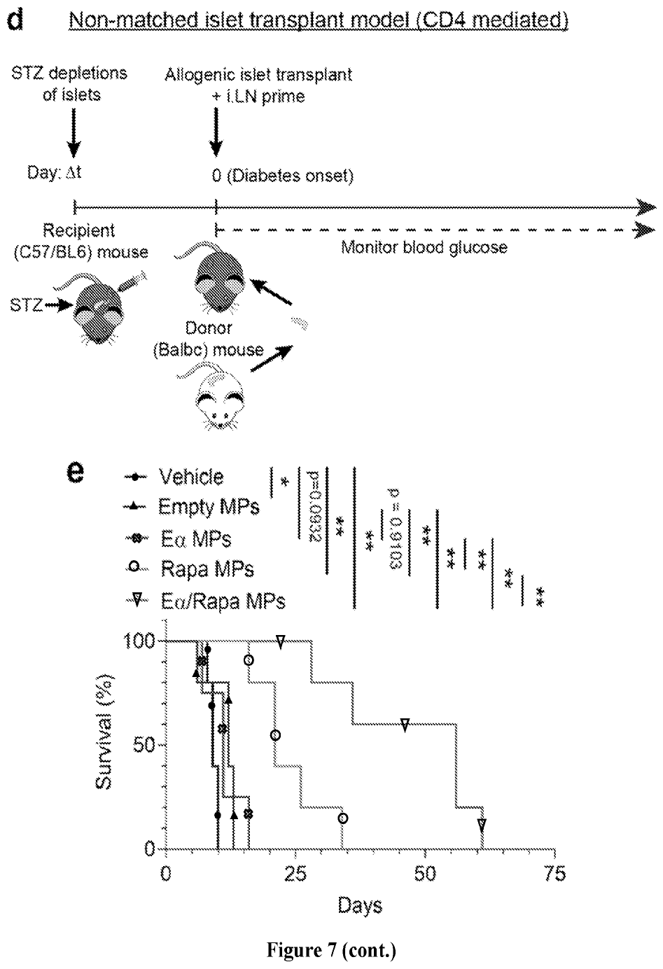

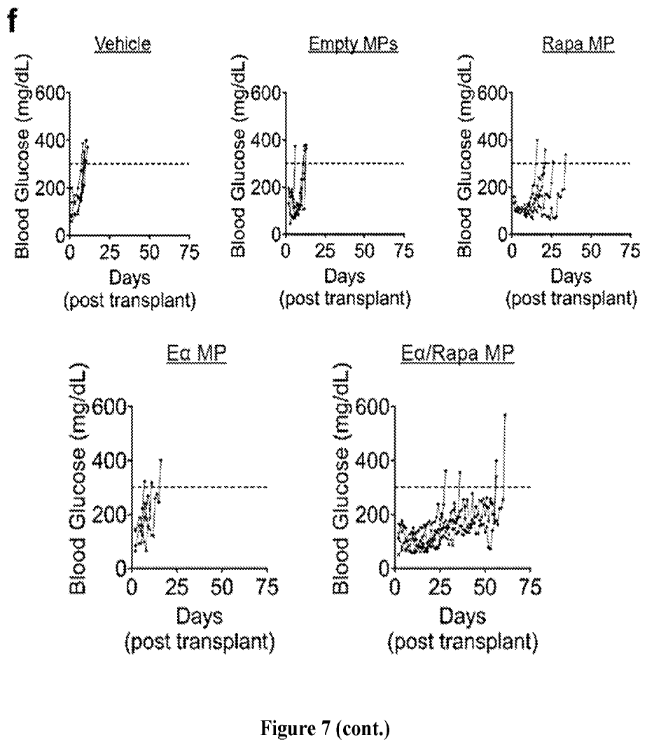

Direct LN injection of tolerogenic MPs promotes tolerance in pre-clinical models of T1D and islet transplantation. As an initial setting to test the tolerizing ability of MPs in T1D, we used an accelerated model of T1D where ex vivo activated NRP-V7 specific T cells are adoptively transferred into NOD mice (FIG. 7a). Mice were treated intra-LN 3 days prior to transfer to test if NPR-V7/rapamycin MPs could stop T1D, and importantly, if MPs loaded with a different disease-relevant peptide (e.g., CgA/rapamycin MPs) could also stop disease. Excitingly, both NRP-V7/rapamycin MP and CgA/rapamycin MPs prevented disease in all animals, whereas CgA-only MPs provided no benefit relative to vehicle (FIG. 7b, 7c). These data indicate inclusion of either a CD4-dominant (CgA) or CD8-dominant (NRP-V7) epitope provided efficacy, even when disease was mediated by CD8 reactivity (e.g., NRP-V7 T cells). We also note rapamycin MPs partially inhibited disease, which may result from broader transient suppression. This treatment was within 3 days of adoptive transfer. In additional studies, more challenging treatment regimens were employed to further investigate the role of each component.

Intra-LN injection of tolerogenic MPs promotes tolerance in T1D and suppression of graft rejection in allogenic islet transplantation. (FIG. 7a) Schematic representation of intra-LN treatment in the NRP-V7 model of T1D. (FIG. 7b) Survival curve with number of mice which developed disease out of the total mice per group indicated in the parenthesis in the legend. (FIG. 7c) Individual blood glucose traces for mice in each group from (FIG. 7b). (FIG. 7d) Schematic representation of intra-LN treatment in the non-matched islet transplant model. (FIG. 7e) Survival curve for experiment detailed in (FIG. 7d). (FIG. 7f) Individual blood glucose traces for mice in each group (FIG. 7e). N=5 mice for all groups except N=4 mice for the rapamycin MP treatment group. Two tailed log-rank (Mantel-Cox) testing was used for all pairwise comparisons between each treatment for survival curves in (FIG. 7b) and (FIG. 7e).

In addition to the NRP-V7 model, we tested the ability of intra-LN MPs to inhibit islet graft rejection in a non-matched transplantation model. Islets were chemically depleted in C57/BL6 mice using streptozotocin (STZ), followed by transplantation of allogenic islets from Balbc mice in the kidney capsule (FIG. 7d). In this model, alloantigen from MHC-II (Eα) of donor islets is targeted by the adaptive immune response. Intra-LN treatment was administered on the first day of disease, indicated by hyperglycemia (FIG. 7d). Vehicle, Empty MP, and Eα MP treated cohorts all rapidly rejected transplanted islets (FIG. 7e), with median survivals of 9, 12, and 11 days, respectively. Similar to the NRP-V7 model of T1D, rapamycin MP alone provided prolonged survival compared to Vehicle or MPs encapsulating Eα antigen alone (FIG. 7e, 7f). Intriguingly, in this model, Eα/rapamycin MPs provided significant therapeutic benefit with a median survival of 56 days vs 21 days and 9 days for the rapamycin MPs and vehicle, respectively. Together, efficacy in these pre-clinical models demonstrates depots potently combat T1D and islet rejection (e.g., substantially delaying and/or preventing graft rejection), and co-encapsulation of rapamycin and disease-relevant—but not necessarily the disease-induction epitope—maximized efficacy.

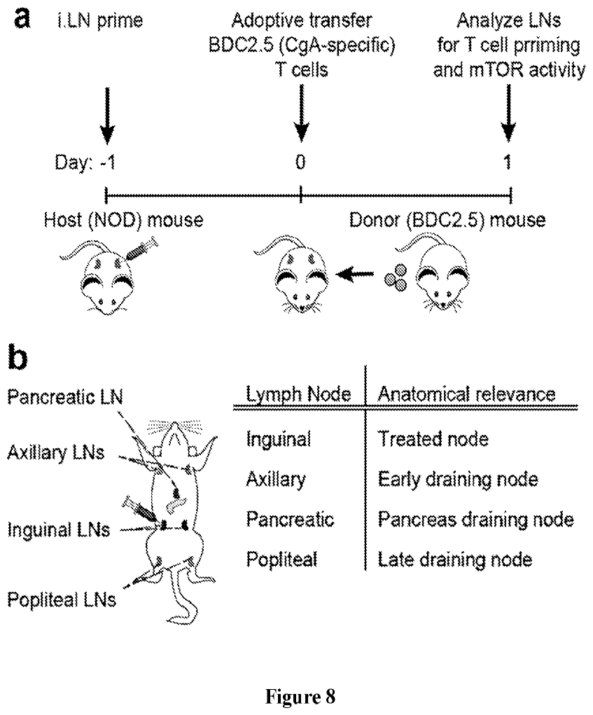

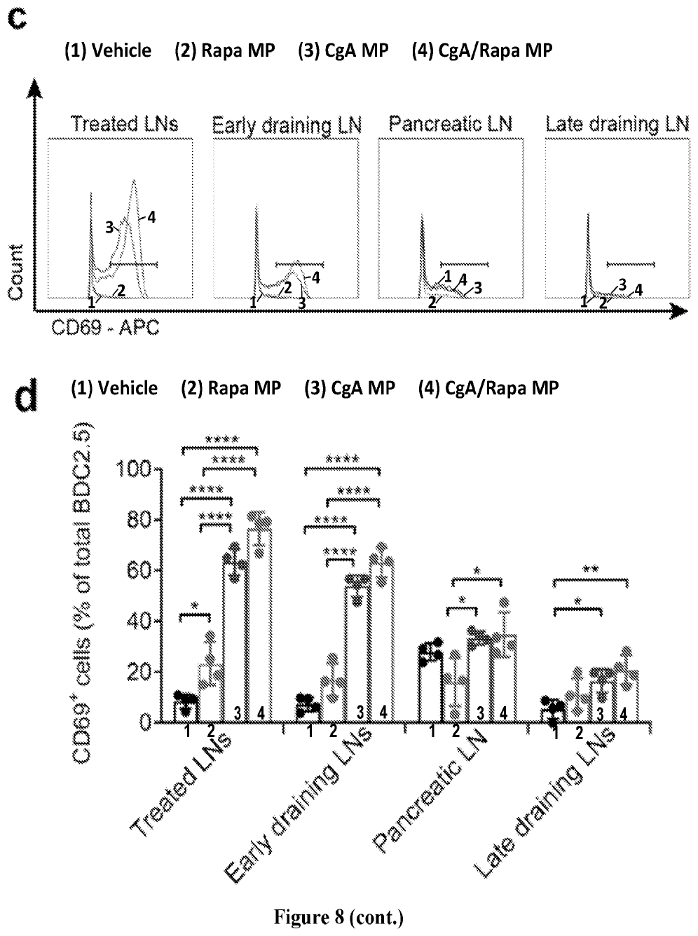

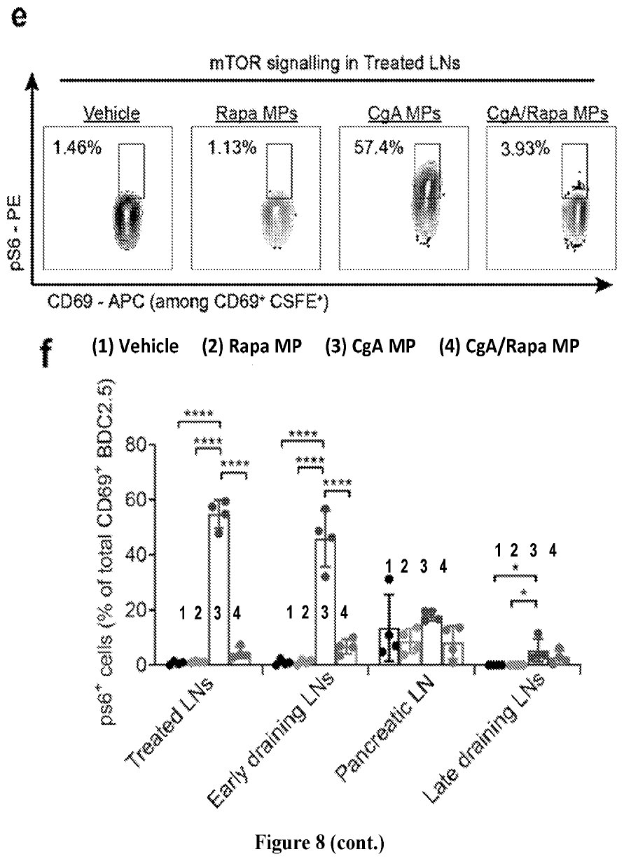

CgA/rapamycin MPs prime antigen-specific T cells in treated and untreated LNs while suppressing mTOR activity. Since MP treatment promotes tolerance with respect to disease pathology, we next investigated the mechanism and localization of T cell polarization by testing if MPs prime antigen-specific T cells and inhibit mTOR in treated and untreated LNs. NOD mice were injected with CgA/rapamycin MPs in the inguinal LNs, and BDC2.5 T cells were adoptively transferred to increase the frequency of antigen-specific T cells for analysis (FIG. 8a). BDC2.5 T cells were labeled with CFSE prior to cell transfer to allow tracking at early timepoints. One day after transfer we isolated the transferred cells from LNs (FIG. 8b) and analyzed them for CD69 and phosphorylated ribosomal s6 protein (ps6) expression. CD69 is an early activation marker upregulated within hours of antigen-specific stimulation, while ps6 correlates with increased mTOR signaling. BDC2.5 T cells in treated LNs of mice receiving MP formulations containing CgA antigen displayed similar levels of CD69 expression, which were elevated compared to cells in treated LNs of mice receiving MP formulations without antigen (FIG. 8c, 8d). Interestingly, this trend was also observed in the early treatment-draining axillary LNs (untreated), and to a lesser extent in the pancreas draining LNs and the distant later-draining popliteal LNs (FIG. 8c, 8d). Concurrently with upregulation of early activation markers, we discovered inclusion of rapamycin in MPs dramatically suppressed phosphorylation of s6 through the mTOR pathway among these cells (FIG. 8e, 8f). Once again, this trend was presented in treated and untreated LNs. Together these data demonstrate CgA/rapamycin MPs prime antigen-specific T cells and modulate mTOR activity, a combination of features that could provide sufficient stimulus for T cell expansion, while polarizing these cells toward T_{REG}.

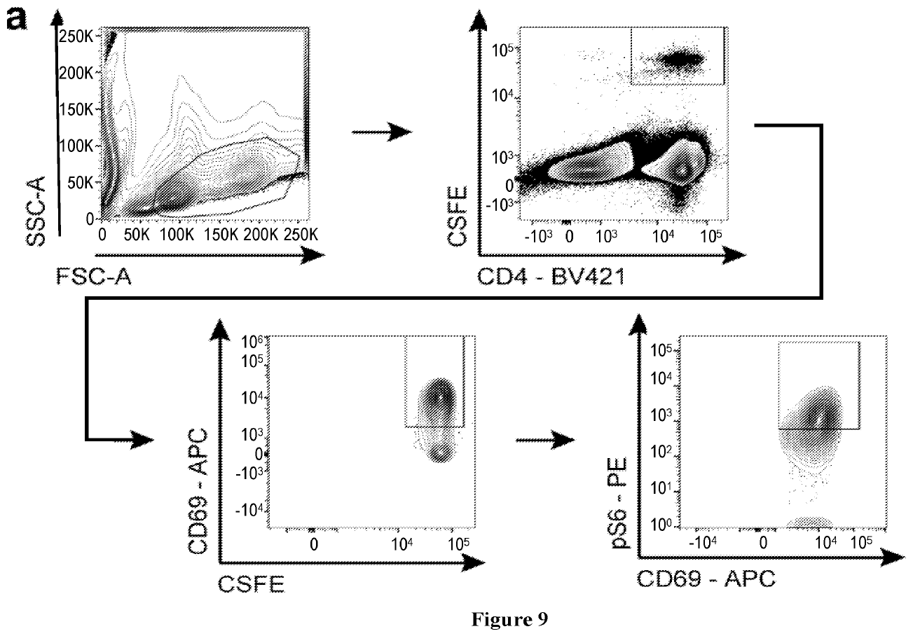

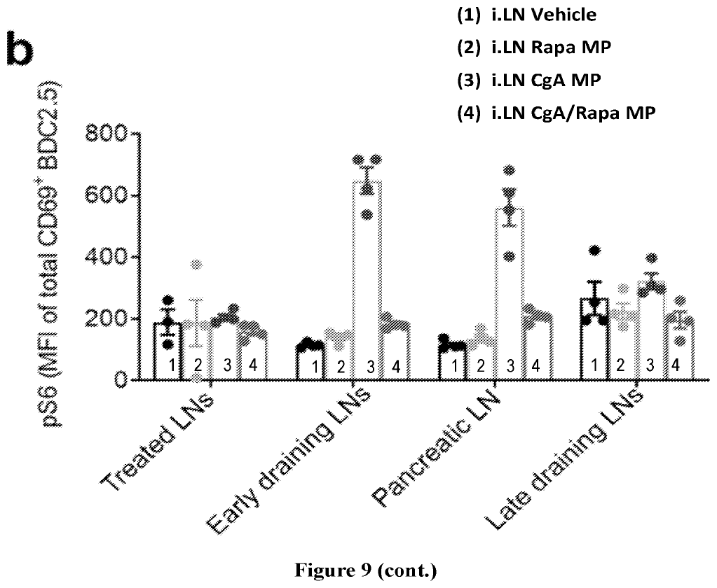

CgA/rapamycin MPs promote antigen-specific T cell priming while inhibiting mTOR in vivo. (FIG. 8a) Schematic representation of MP treatment and experimental readout for BDC2.5 T cell adoptive transfer experiment. (FIG. 8b) Illustration of lymph nodes collected and their anatomical relevance to treatment. (FIG. 8c) Representative flow cytometry traces and (FIG. 8d) quantification of CD69 expression among CgA specific T cells. (FIG. 8e) Representative flow cytometry traces of phosphorylated s6 among CD69⁺ CgA specific T cells in treated LNs and (FIG. 8f) quantification in all LNs. N=4 mice for all experiments, and paired LNs were pooled for each mouse. The mean±s.d. is shown. One way ANOVA with Tukey's post hoc testing was used to compare each treatment within individual lymph nodes. FIG. 9a illustrates the gating scheme to analyze CD69 and ps6 expression among CgA specific T cells in the experiment detailed in FIG. 8. Geometric mean fluorescence intensity of ps6 among CgA specific CD69⁺ cells is shown in FIG. 9b.

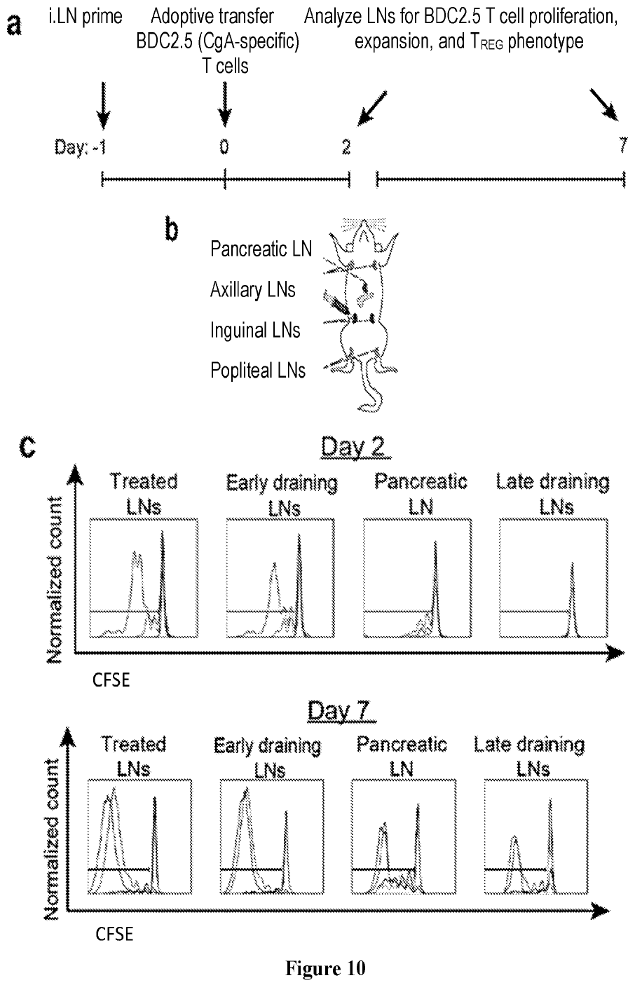

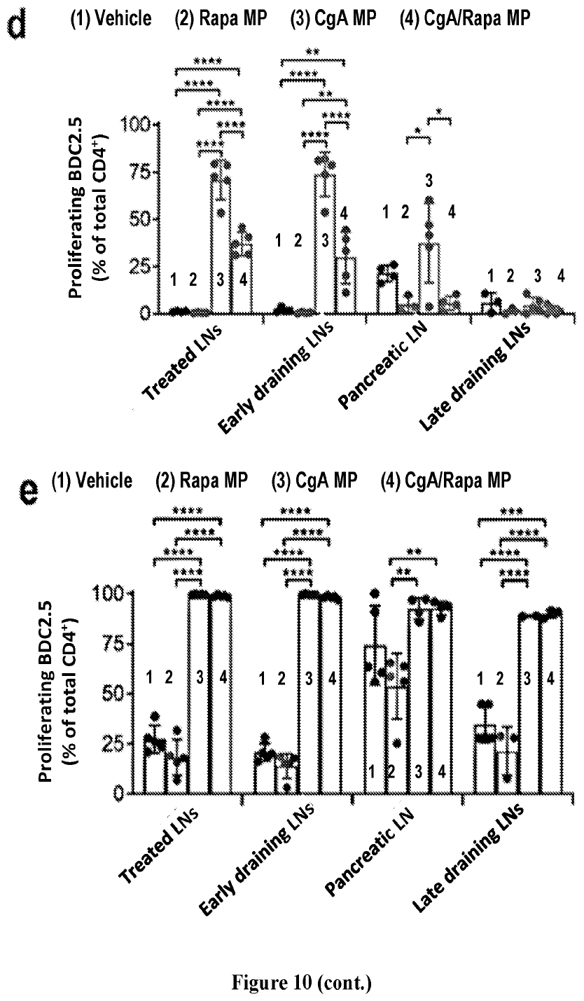

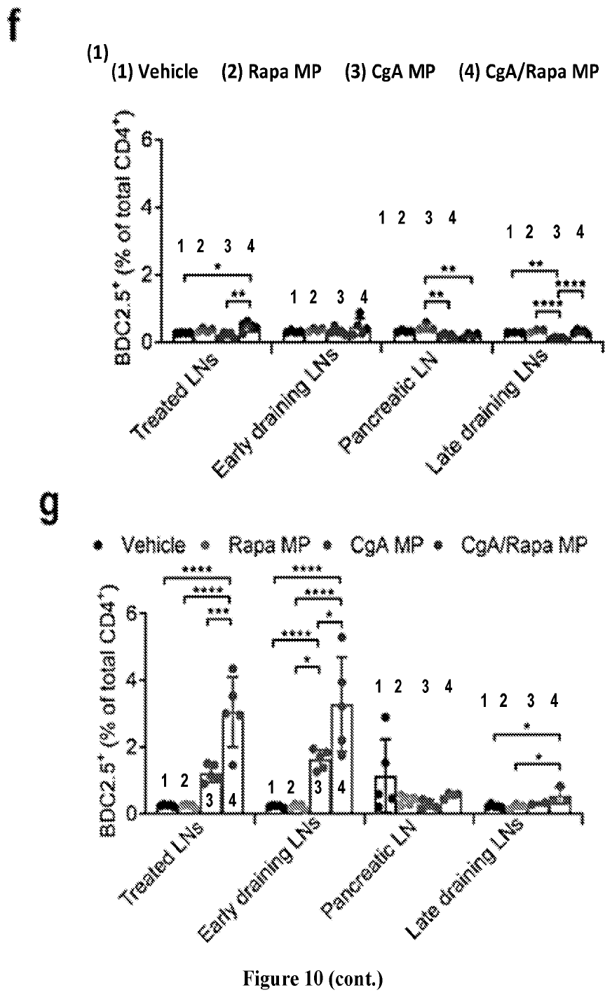

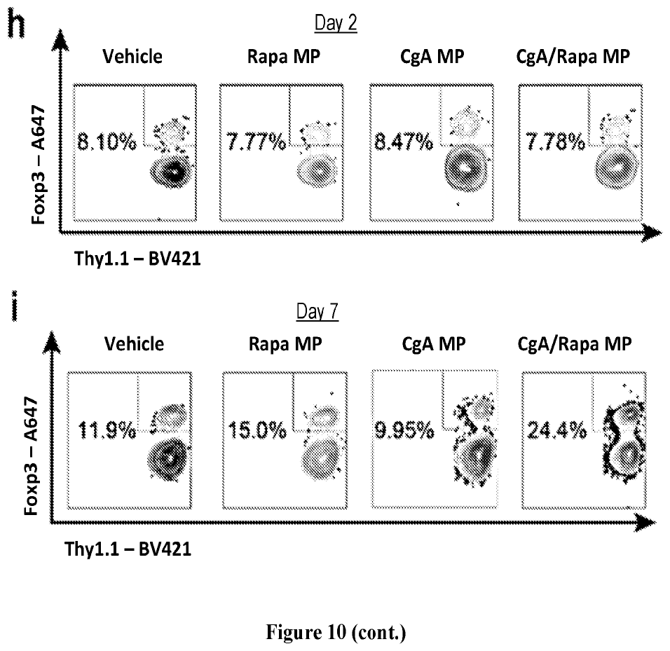

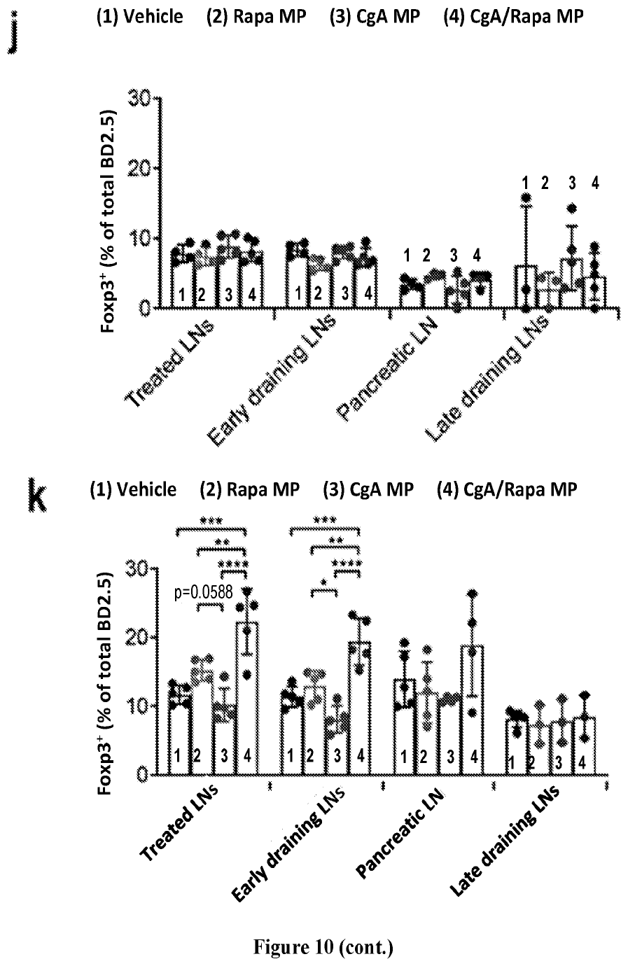

MPs expand antigen-specific T_{REG} in treated and untreated LNs. To investigate, we tested if MP treatment expands antigen-specific T_{REG} by again treating NOD mice intra-LN with MPs and transferring BDC2.5 T cells (FIG. 10a). 2 and 7 days after T cell transfer, treated and non-treated LNs were isolated (FIG. 10b). In this experiment T cells from BDC2.5 mice with a thy1.1 marker were used to enable tracking at later timepoints when CFSE may be diluted to undetectable levels. At 2 days post transfer, while both CgA MPs and CgA/rapamycin MPs induced significant BDC2.5 T cell proliferation (FIG. 10c, 10d), the frequency of proliferated cells was significantly higher in the treated, early-draining, and pancreatic LNs of mice treated with CgA MP relative to CgA/rapamycin MP treatment (FIG. 10d). 7 days post transfer, nearly all BDC2.5 T cells in mice treated with CgA or CgA/rapamycin MPs had proliferated in each LN examined, including distant, later-draining LNs (FIG. 10c, 10e). In each case, the frequency of proliferated T cells was significantly higher in CgA MP or CgA/rapamycin MP-treated mice compared to vehicle or rapamycin MP treated mice in each LN. At day 2, the total frequency of BDC2.5 T cells in LNs among all CD4 T cells was low (<0.5% for each group in each LN) (FIG. 10). By day 7, expansion of BDC2.5 T cells increased in both treated and draining LNs of mice receiving CgA/rapamycin MPs and CgA MPs, but intriguingly, CgA/rapamycin MPs caused significantly greater increases than CgA MPs (FIG. 10g). Finally, while analysis of Foxp3 expression showed no differences among BDC2.5 T cells at day 2 in any LN (FIG. 10h, 10j), at day 7, CgA/rapamycin MP treatment dramatically expanded T_{REG} among antigen-specific T cells in treated and draining LNs compared to all treatment groups (FIG. 10i, 10k). These data demonstrate the presence of CgA is beneficial to expand antigen-specific T cells in treated and untreated LNs, and inclusion of rapamycin is beneficial to polarize antigen-specific T cells to T_{REG}.

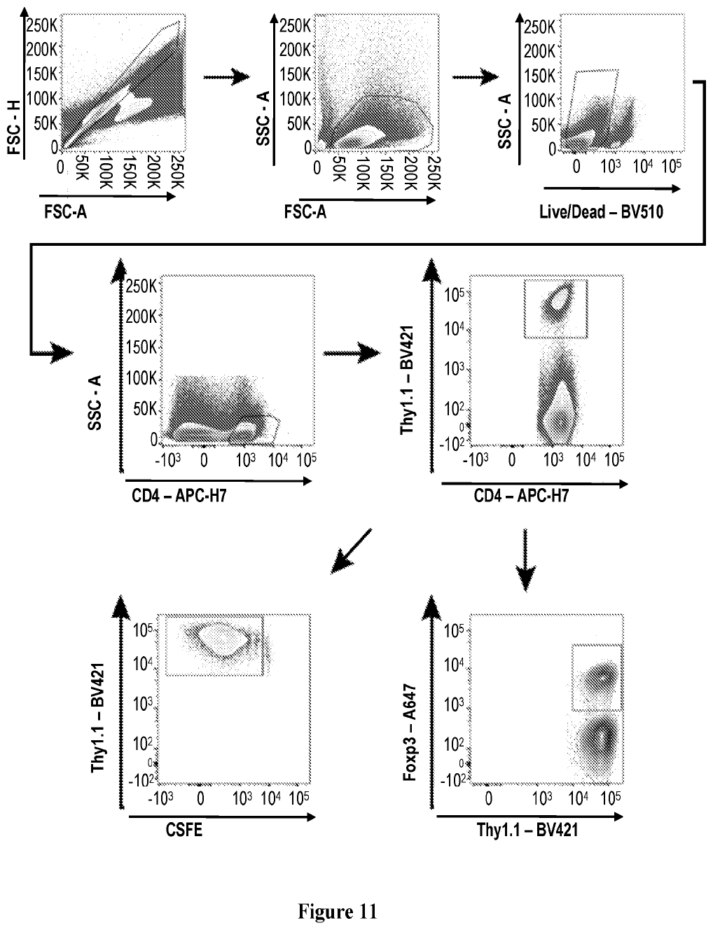

CgA/rapamycin MPs expand antigen-specific T_{REG} in treated and untreated LNs. (FIG. 10a) Schematic representation of MP treatment and experimental readout for BDC2.5 T cell adoptive transfer experiment. (FIG. 10b) Illustration of LNs collected and analyzed. (FIG. 10c) Representative flow cytometry traces and quantification of CgA specific T cell proliferation at (FIG. 10d) day 2 and (FIG. 10e) day 7. Quantification of CgA specific T cell frequency at (FIG. 10f) day 2 and (FIG. 10g) day 7. Representative flow cytometry traces showing Foxp3 expression among CgA specific T cells in treated LNs at (FIG. 10h) day 2 and (FIG. 10i) day 7. Quantification of Foxp3 expression among CgA specific T cells at (FIG. 10j) day 2 and (FIG. 10k) day 7 in each LN analyzed. N=5 mice for all groups in all experiments, and paired LNs were pooled for each mouse. The mean±s.d. is shown. One way ANOVA with Tukey's post hoc testing was used to compare each treatment within individual lymph nodes. The gating scheme is shown in FIG. 11 to analyze Foxp3 expression and CFSE dilution among CgA specific T cells in the experiments detailed in FIG. 10.

Previous work in multiple models of autoimmune disease has demonstrated particulate delivery of antigens alone as an effective strategy to promote antigen-specific tolerance (Gosselin, E. A. et al. (2018) Designing natural and synthetic immune tissues. Nat. Mater. 17, 484-498; Saito, E. et al. (2020) Modulating lung immune cells by pulmonary delivery of antigen-specific nanoparticles to treat autoimmune disease. Sci. Adv. 6, eabc9317; Shah, S. et al. (2019) Optimizing PLG nanoparticle-peptide delivery platforms for transplantation tolerance using an allogeneic skin transplant model. Biomaterials 210, 70-82; Hunter, Z. et al. (2014) A Biodegradable Nanoparticle Platform for the Induction of Antigen-Specific Immune Tolerance for Treatment of Autoimmune Disease. ACS Nano 8, 2148-2160; Getts, D. R. et al. (2014) Therapeutic Inflammatory Monocyte Modulation Using Immune-Modifying Microparticles. Sci. Transl. Med. 6, 219ra7; Hess, K. L. et al. (2017) Engineering Immunological Tolerance Using Quantum Dots to Tune the Density of Self-Antigen Display. Adv. Funct. Mater. 27, 1700290; Carambia, A. et al. (2015) Nanoparticle-based autoantigen delivery to Treg-inducing liver sinusoidal endothelial cells enables control of autoimmunity in mice. J. Hepatol. 62, 1349-1356). The mechanisms involved in these studies generally leverage antigen processing through non-inflammatory pathways by APCs in LNs, spleens and liver to restrain antigen-specific immune response. Due to activation of these tolerogenic pathways, these treatments do not require inclusion of a regulatory immune cue, functionally delete or exhaust auto-antigen-specific T cells, and promote active tolerance through the generation of T_{REG}. Previous studies have shown particulate delivery of self-antigen promotes tolerance in EAE after systemic administration of antigen conjugated to the surface of quantum dot nanoparticle (Hess, K. L. et al. (2017) Engineering Immunological Tolerance Using Quantum Dots to Tune the Density of Self-Antigen Display. Adv. Funct. Mater. 27, 1700290), or when using the intra-LN MP platform (Tostanoski, L. H. et al. (2016) Reprogramming the Local Lymph Node Microenvironment Promotes Tolerance that Is Systemic and Antigen Specific. Cell Rep. 16, 2940-2952). The latter study demonstrated co-encapsulation with rapamycin promoted the greatest therapeutic benefit (in agreement with the results presented here). These data indicate mechanistically antigen is beneficial for activation and expansion of antigen-specific T cells, and rapamycin is beneficial to drive regulatory phenotype. Interestingly, intra-LN treatment with CgA/rapamycin MPs does not exhaust antigen-specific T cells, but promotes active tolerance which may provide durable control of autoimmune reactions.

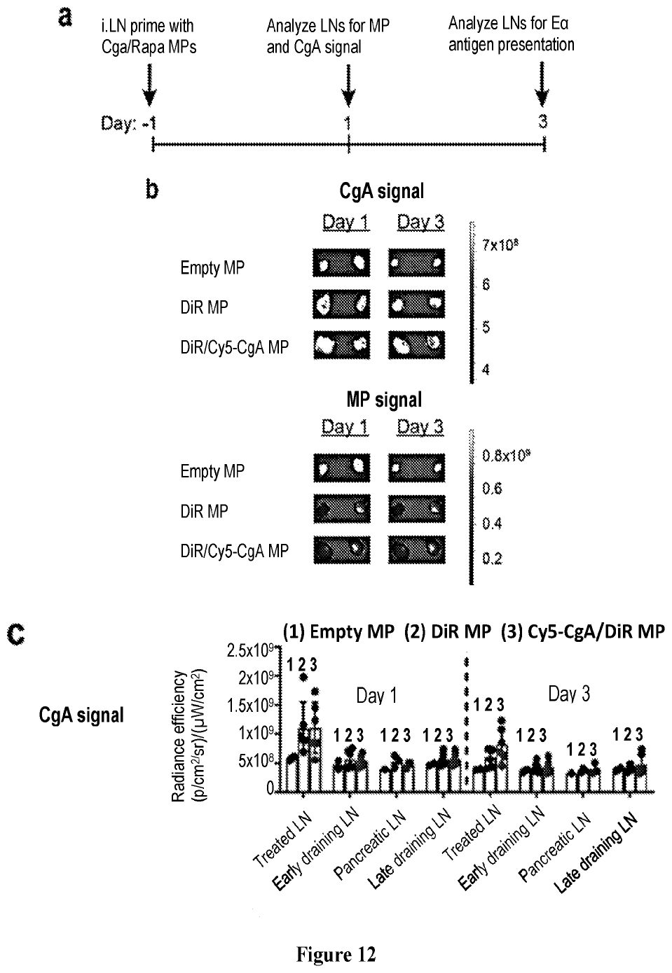

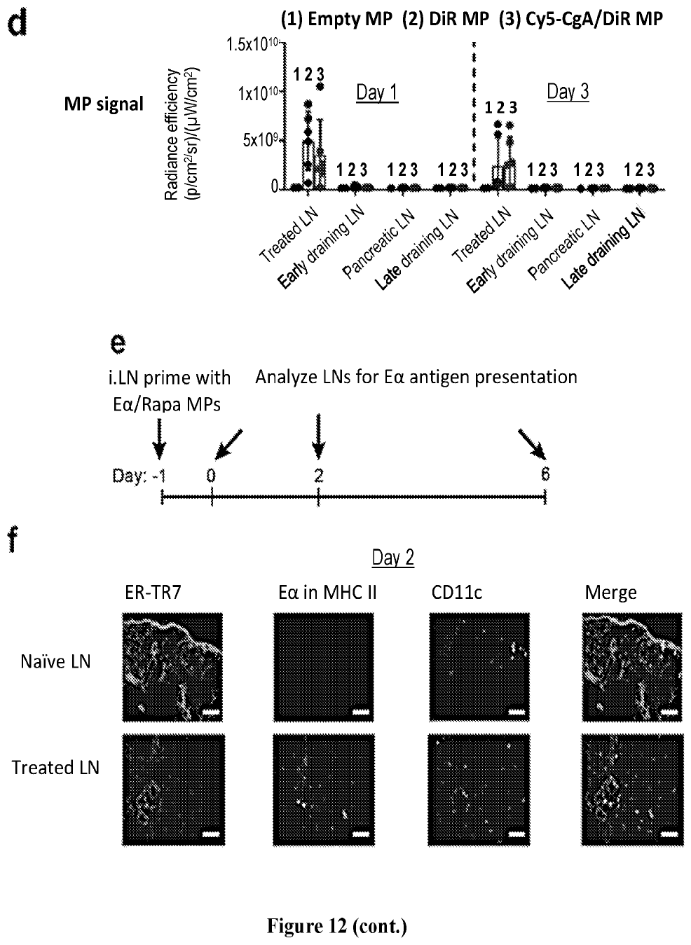

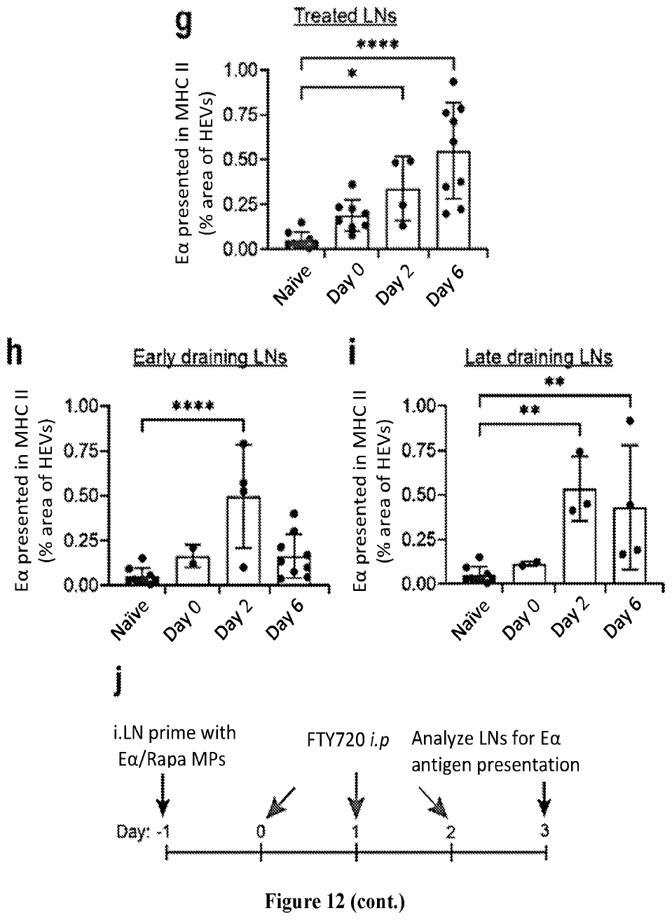

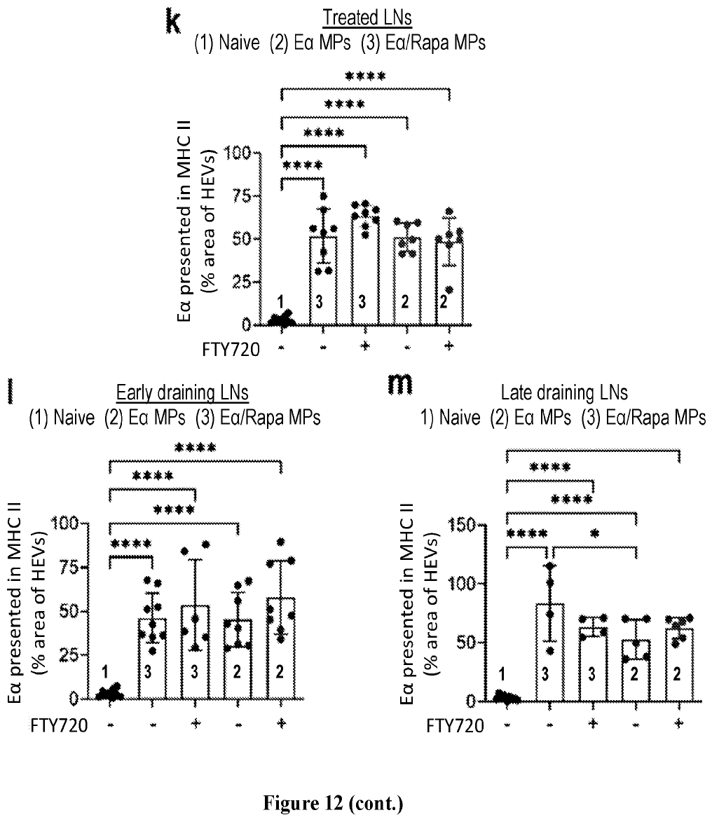

Antigen delivered in MPs is presented in treated and untreated LNs, and presentation in untreated LNs is independent of antigen presenting cell (APC) migration. The finding that CD69—which is transiently upregulated upon stimulation and facilitates temporary retention in LNs (Cibrián, D. & Sánchez-Madrid, F. (2017) CD69: from activation marker to metabolic gatekeeper. Eur. J. Immunol. 47, 946-953)—is upregulated in both treated and non-treated LNs, suggests antigen is present in these sites. Since these MPs are size restricted and do not drain through efferent lymphatics, there are two likely mechanisms for delivery to LNs: drainage through lymphatics after release from MPs or trafficking by APCs after internalization of MPs. To confirm that MPs are retained in treated LNs, mice were injected intra-LN with unlabeled MPs, MPs encapsulating a fluorescent dye, DiR (DiR MPs), and MPs co-encapsulating FITC-labeled CgA and DiR (FITC-CgA/DiR MPs) (FIG. 12a). 2 and 4 days after MP treatment, treated, draining and pancreatic LNs were isolated and imaged by IVIS for DiR and FITC-CgA signal (FIG. 12b). We did not detect DiR and FITC signals in any of the LNs receiving unlabeled MP formulations (FIG. 12c, 12d). However, there was a significantly increased DiR signal in LNs treated with DiR MPs and DiR/FITC-CgA MPs at both 2 and 4 days after treatment. In these groups DiR signal was only detectable in treated LNs, supporting local MP retention in treated LNs (FIG. 12c, 12d; purple). The same phenomenon was observed in mice receiving FITC-CgA/rapamycin MPs (FIG. 12b, 12d; red). These data demonstrate restriction of large cargo reservoirs to the treated LNs using intra-LN depots, but the possibility remains that antigen is undetectable at the macroscopic level by IVIS drains to other LNs and is sufficient to promote T cell priming.

Subsequently, we directly investigated the trafficking and presentation of antigen from MPs in untreated LNs using Y-ae, a recently-developed antibody tool. Y-ae binds to the Eα epitope when presented in MHC-II (Warren, K. J. et al. (2014) Laminins affect T cell trafficking and allograft fate. J. Clin. Invest. 124, 2204-2218). To study Eα presentation, mice were immunized intra-LN with Ea/rapamycin MPs on day 1, then the treated and untreated LNs were stained for Eα presentation in areas of LNs surrounding high endothelial venules (HEVs) on days 0, 2, and 6 (FIG. 12e). HEVs are vessels in the cortical ridge (CR) which are localized with lymphatic vessels (Li, L. et al. (2020) The lymph node stromal laminin α5 shapes alloimmunity. J. Clin. Invest. 130:2602-2619). Since these regions create beneficial networks where antigens, APCs/DCs, and T cells interface to initiate and direct antigen-specific immune processes (Burrell, B. E. et al. (2015) Lymph node stromal fiber ER-TR7 modulates CD4+ T cell lymph node trafficking and transplant tolerance1. Transplantation 99, 1119-1125), we investigated presentation of MP delivered Eα at these sites in treated and untreated LNs. At 2 days, Eα presentation localized with HEVs was detected in treated LNs (FIG. 12f), and increased by day 6 (FIG. 12g). Likewise, at day 2, Eα presentation localized with HEV was also detected in the early-draining (FIG. 12h) and late-draining (FIG. 12i) LNs; by 6 days only the signal in the late-draining LNs remained elevated (FIG. 12h, 12i).

Since antigen presentation was confirmed in untreated LNs after intra-LN injection, we next tested if antigen trafficking may be through lymphatic drainage following release from MPs, or through migration of APCs that internalize MPs. Mice were immunized with Eα MPs or Eα/rapamycin MPs, and then APC egress from LNs was inhibited by administration of FTY720 (Honig, S. M. et al. FTY720 stimulates multidrug transporter—and cysteinyl leukotriene dependent)—an antagonist of the S1p receptor, using three daily i.p. injections starting on the day of MP treatment (FIG. 12j). 1 day after the final FTY720 injection, treated and draining LNs were analyzed for Eα presentation in HEVs. In every LN analyzed, Eα and Eα/rapamycin MP treatment significantly increased Eα presentation in treated (FIG. 12k), early-draining (FIG. 12l) and late-draining LNs (FIG. 12m) compared to the naïve control. FTY720 treatment did not significantly affect Eα presentation (FIG. 12k, 12l, 12m) for either MP treatment, indicating trafficking of antigen is not reliant on migration of APCs from treated LNs.

Thus, antigen delivered in MPs drains to and is presented in untreated LNs and drainage is independent of APC migration. After intra-LN injection of CgA/rapamycin MPs, MPs are retained in treated LNs, while CgA is released from MPs and drains to untreated LNs. After injection with Eα/rapamycin MPs, Eα is presented in MHC in treated and untreated LNs. After injection with Eα/rapamycin MPs, Eα trafficking to other LNs is independent of APC migration via S1p receptor. The data demonstrate that antigen is presented in untreated LNs after MP treatment, showing migration of antigen-specific T cells to untreated LNs after MP treatment. In addition, the data demonstrate that antigen expression in untreated LNs which is independent of APC migration is novel.

Ea is presented in treated and untreated LNs after Ea/rapamycin MP treatment. (FIG. 12a) Schematic representation of experiment in FIG. 12b-d. N=3 mice for DiR MP and FITC-CgA/DiR MP treatment groups and N=1 mouse for empty MP treatment group at each timepoint. Each data point is represented as an individual data point. (FIG. 12b) IVIS images of excised LNs in treated LNs. (FIG. 12c) Quantitation of MP signal and (FIG. 12d) Cy5-CgA signal in excised LNs. One way ANOVA with Tukey's post hoc testing was used to compare each treatment group, at each time point. Individual LNs were used as single data points. Mean±s.d. is shown. (FIG. 12e) Schematic representation of experiment (FIG. 12f-i). N=2 mice for each timepoint. (FIG. 12f) Representative immunofluorescent images of LNs stained with ER-TR7, CDTic, YAe; Scale bar=50 μm. Quantification of Eα presentation in MHC-II (YAe) in (FIG. 12g) treated, (FIG. 12h) early-draining and (FIG. 12i) late-draining LNs. One way ANOVA with Tukey's post hoc testing was used to compare each indicated LNs at each timepoint. (FIG. 12j) Schematic representation of experiment (FIG. 12k-m). Quantification of Eα presentation in MHC-II in (FIG. 12k) treated, (FIG. 12l) early-draining, and (FIG. 12m) late-draining LNs after FTY720 treatment. One way ANOVA with Tukey's post hoc testing was used to compare each group. Mean±s.d. is shown for each sample in each experiment.

Inhibition of mTOR signaling in untreated tissues of CgA/rapamycin MP treated mice (FIG. 10b, 10c) indicates dissemination of rapamycin to these LNs, similar to antigen. Exposure of multiple non-treated LNs to antigen and rapamycin following MP treatment may enhance antigen-specific T_{REG} expansion and therapeutic efficacy. As demonstrated in other studies, the co-delivery of immunostimulatory adjuvants and antigen using the intra-LN platform with MPs promoted superior antigen-specific T cell response, as compared to treatment with nanoparticles (NPs) sufficiently small to drain through efferent lymphatics (Jewell, C. M. et al. (2011) In situ engineering of the lymph node microenvironment via intranodal injection of adjuvant-releasing polymer particles. Proc. Natl. Acad. Sci. 108, 15745-15750). Therefore, it is believed that prolonged retention of delivered cargo in treated LNs after MP treatment is a primary driver for induction of tolerance. An additional key advantage of the intra-LN platform of the present disclosure is the ability to deliver the entire dosage of immune signals to the lymphatic system in order to promote antigen-specific T_{REG} in LNs without exposing peripheral tissue to immunosuppressants. While particulate delivery of antigen can enter the lymphatics and reach multiple LNs after peripheral administration, a large fraction of payload is retained at the injection site, or can reach the non-lymphoid tissues by engineering particle size and properties (Johansen, P. et al. (2008) Antigen kinetics determines immune reactivity. Proc. Natl. Acad. Sci. U.S.A 105, 5189-5194; Almeida, J. P. M. et al. (2011) In vivo biodistribution of nanoparticles. Nanomed. 6, 815-835).

LN delivery of MPs promotes a tolerogenic LN microenvironment. During tolerance induction, structural changes in LNs develop, characterized by increases in the ratio of laminin α4 (lamα4) to laminin α5 (lama5) that are observed locally in the HEVs and CRs of LNs (Simon, T. et al. (2019) Differential Regulation of T Cell Immunity and Tolerance by Stromal Laminin Expressed in the Lymph Node. Transplantation 103, 2075-2089; Warren, K. J. et al. (2014) Laminins affect T cell trafficking and allograft fate. J. Clin. Invest. 124, 2204-2218; Simon, T. & Bromberg, J. S. (2017) Regulation of the Immune System by Laminins. Trends Immunol. 38, 858-871). At these regions, higher lama4:lama5 ratio promotes T_{REG} induction and accumulation, while lower ratios favor differentiation of T_{H}1 and T_{H}17 phenotypes (Simon, T. et al. (2019) Differential Regulation of T Cell Immunity and Tolerance by Stromal Laminin Expressed in the Lymph Node. Transplantation 103, 2075-2089).

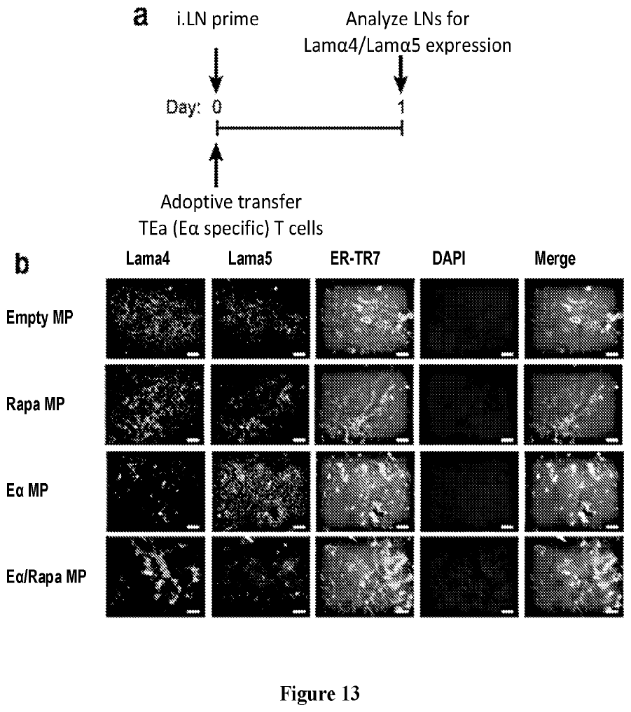

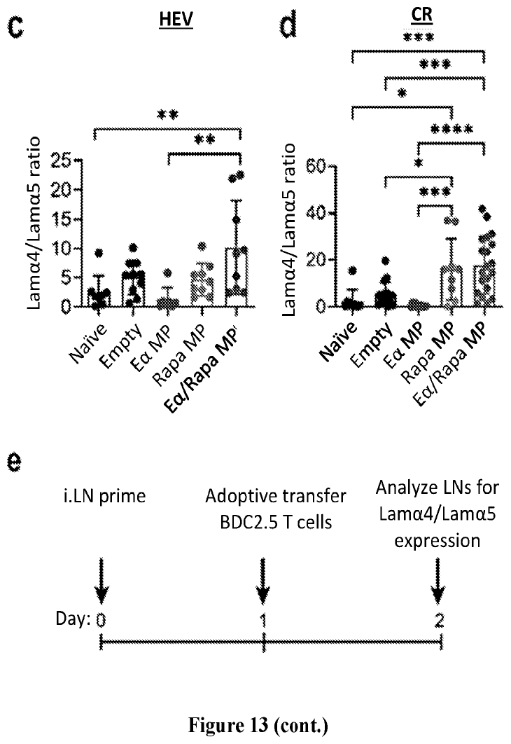

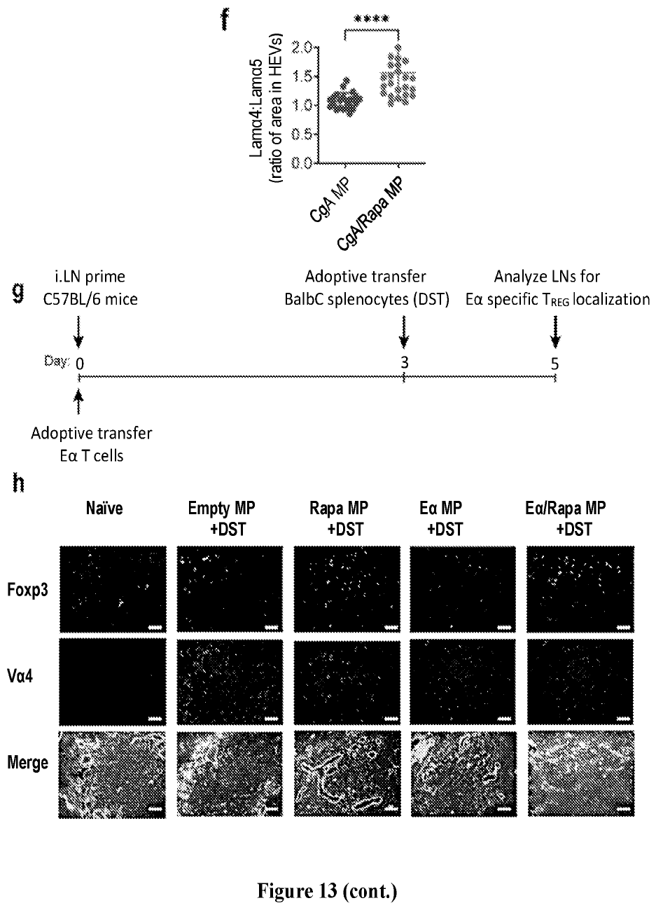

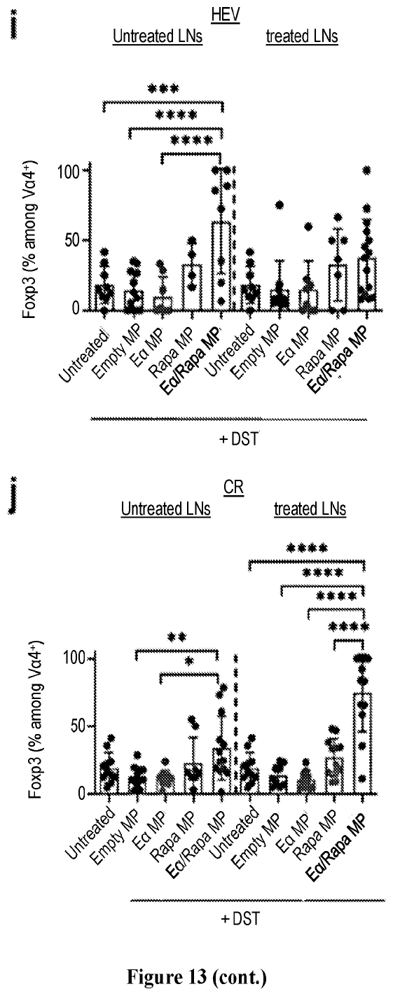

To investigate if local delivery of self-antigen and rapamycin promotes tolerogenic microdomains in treated LNs that promote local T_{REG} polarization, mice were immunized with Eα/rapamycin MPs followed by adoptive transfer of Eα-specific T cells. One day after T cell transfer, treated LNs were analyzed for lama4 and lama5 expression in HEVs and CR (FIG. 13a). In both HEVs (FIG. 13b) and CR (FIG. 13b), Eα/rapamycin MPs promoted the highest lama4:lama5 ratio. Similar results were observed in NOD mice treated with CgA/rapamycin MPs and infused with BDC2.5 T cells (FIG. 13e), where ama4:lama5 ratios were significantly increased in HEVs of pooled LNs when comparing CgA/rapamycin and CgA treatment (FIG. 13f). This data demonstrates co-treatment with rapamycin and antigen—either CgA or Eα—promotes structural changes associated with regulatory T cell response.

Structural and cellular reorganization in LNs during tolerance induction were demonstrated via intra-LN injection of the disclosed MP platforms in accordance with the present disclosure, thereby generating tolerance in LNs after MP treatment. In particular, intra-LN injection of antigen/rapamycin MPs promoted tolerogenic microdomains in LNs, characterized by increased lama4:lama5 ratios and accumulation of antigen specific T_{REG} in HEVs and cortical ridges.

MP treatment promotes development of tolerogenic microdomains in LNs. (FIG. 13a) Schematic representation of experiment in panels b-d. N=4 mice for each treatment group. (FIG. 13b) Representative immunofluorescence images of treated LNs stained for laminin α4, laminin α5, ER-TR7 and DAPI are shown; Scale bar=50 μm. Quantification of ratio of laminin α4 to laminin α5 in (FIG. 13c) high endothelial venules and (FIG. 13d) cortical ridges in treated LNs. One way ANOVA with Tukey's post hoc testing was used to compare treatment groups. (FIG. 13e) Schematic representation of experiment in panel f. N=4 mice for each treatment group. Inguinal, axillary, pancreatic and popliteal LNs were pooled for each treatment group. (FIG. 13f) Ratio of laminin α4 to laminin α5 in high endothelial venules of LNs. Two tailed Mann Whitney testing was used to compare both treatment groups. (FIG. 13g) Schematic representation of experiments in panels i-1. N=4 mice for each treatment group. (FIG. 13h) Representative immunofluorescence images showing Foxp3 expression among antigen-specific T cells. DAPI (Blue) and ER-TR7 (Cyan) are included in the merge: Scale bar=50 μm. LNs were stained for Foxp3, Vα4 T cell receptor clonotype, ER-TR7, and DAPI. Quantification of Foxp3 expression among antigen-specific T cells in (FIG. 13i) high endothelial venules and (FIG. 13j) cortical ridges. One way ANOVA with Tukey's post hoc testing was used to compare each treatment group within individual lymph nodes in each region of the LN analyzed. For all experiments mean±s.d. is shown.

To determine if intra-LN MPs promote local accumulation of antigen-specific T_{REG} in HEVs and CRs, C57/BL6 mice were immunized with Ea/rapamycin MPs, followed by adoptive transfer of Eα T cells. 3 days after T cell transfer, non-matched BalbC donor-specific splenocytes (DST) were adoptively transferred to generate a systemic inflammatory alloimmune response (Warren, K. J. et al. (2014) Laminins affect T cell trafficking and allograft fate. J. Clin. Invest. 124, 2204-2218). 2 days following DST infusion, LNs were analyzed for T_{REG} accumulation in HEVs and CRs. To identify antigen-specific T_{REG}, LNs were co-stained for Foxp3 and the Vα4 T cell receptor chain, which is expressed by Eα T cells (Warren, K. J. et al. (2014) Laminins affect T cell trafficking and allograft fate. J. Clin. Invest. 124, 2204-2218). Eα/rapamycin MPs promoted the highest increase of antigen-specific T_{REG} in HEVs and CRs (FIG. 13h, 13i, 13j). Together these data indicate self- or auto-antigen/rapamycin MPs promote tolerogenic microdomains in LNs in both homeostatic and inflammatory conditions.

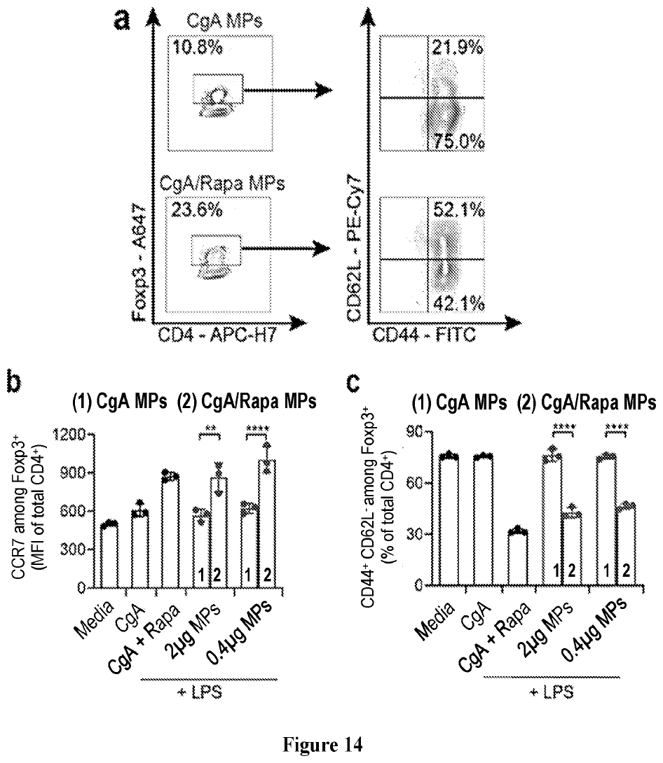

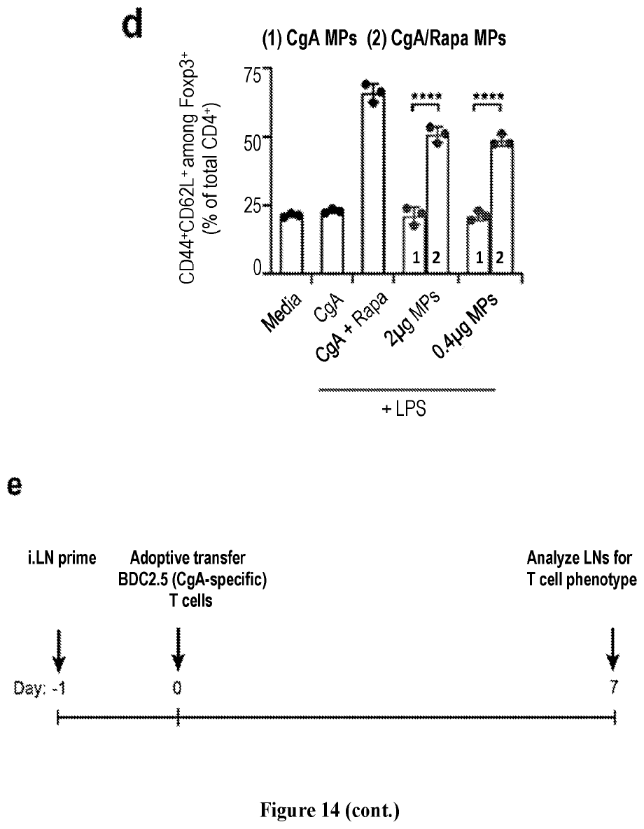

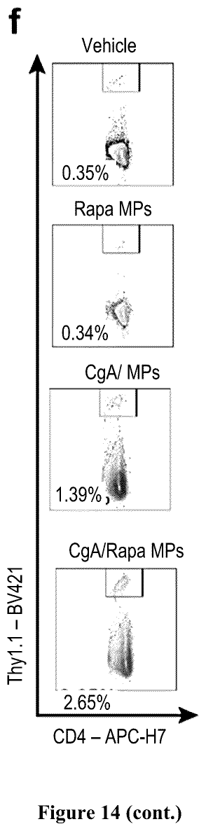

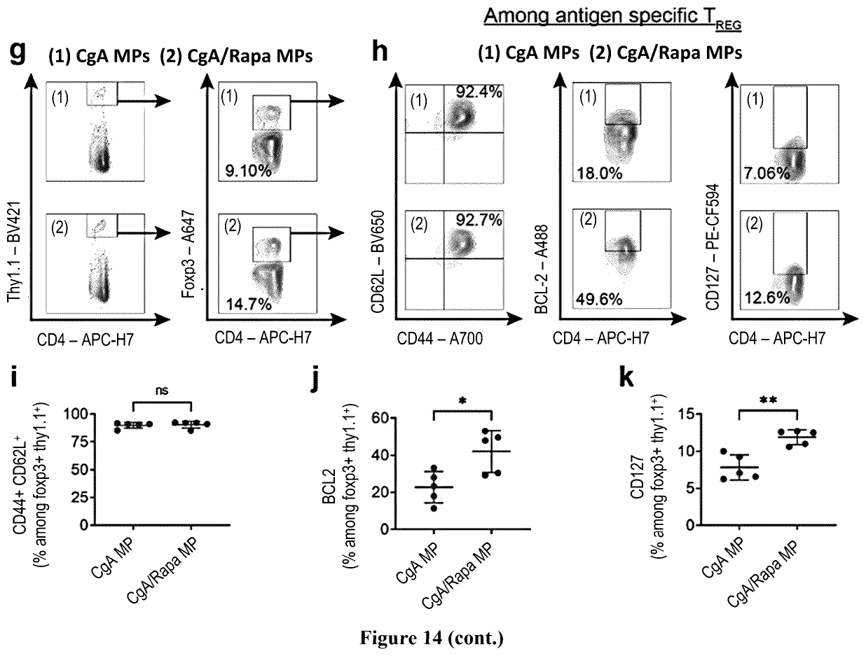

MPs expand antigen-specific T_{REG} with increased expression of markers of longevity. Studies have demonstrated mTOR pathway inhibition during expansion of naïve T cells polarizes central memory phenotype among T_{REG}, which display increased persistence in vivo (Sun, I.-H. et al. (2018) mTOR Complex 1 Signaling Regulates the Generation and Function of Central and Effector Foxp3+ Regulatory T Cells. J. Immunol. 201(2):481-492). Since antigen presentation after intra-LN treatment (FIG. 12) was prolonged we posited that T_{REG} responses which are polarized during mTOR inhibition may also be durable or adopt memory capacity. To test whether inclusion of rapamycin in MPs enhances memory-like phenotype of self-reactive T_{REG}, we first co-cultured DCs from NOD mice with BDC2.5 T cells in the presence of CgA MPs or CgA/rapamycin MPs. After 3 days T cells were stained for Foxp3 and markers characteristic of central memory T cells: CD44⁺ CD62L⁺ and CCR7. CD44 is a marker expressed by activated T cells, while CD62L and CCR7 drive homing to LNs, where long lived memory T cells persist (Catron, D. M. et al. (2006) CD4+ T cells that enter the draining lymph nodes after antigen injection participate in the primary response and become central-memory cells. J. Exp. Med. 203, 1045-1054; Klebanoff, C. A. et al. (2005) Central memory self/tumor-reactive CD8+ T cells confer superior antitumor immunity compared with effector memory T cells. Proc. Natl. Acad. Sci. U.S.A 102, 9571-9576). Excitingly, co-cultures treated with CgA/rapamycin MPs increased the frequencies of T_{REG} co-expressing CD62L and CD44 (FIG. 14a, 14b, 14c), as well as CCR7 (FIG. 14d). These results demonstrate inclusion of rapamycin in MPs promotes a T_{REG} response with central memory phenotype in vitro.

We next analyzed the effects of CgA/rapamycin MP treatment on the expression of central memory markers among antigen-specific T_{REG} in vivo. NOD mice were immunized with CgA/rapamycin MPs, followed by adoptive transfer of BDC2.5 T cells (FIG. 14e). One week after transfer, LNs were analyzed for expression of CD44, CD62L, as well as CD127, and BCL-2 among antigen-specific Foxp3+T_{REG} (FIG. 14e) in treated LNs. CD127 is the receptor for IL-7, an important survival signal for memory T cells within LNs, and BCL-2 functions to inhibit apoptosis (Wojciechowski, S. et al. (2007) Bim/Bcl-2 balance is critical for maintaining naive and memory T cell homeostasis. J. Exp. Med. 204(7):1665-1675; Schluns, K. S. et al. (2000) Interleukin-7 mediates the homeostasis of naïve and memory CD8 T cells in vivo. Nat Immunol 1:426-432). LNs treated with CgA/rapamycin MPs displayed significantly higher frequencies of antigen-specific T cells 1 week after adoptive transfer in the CgA/rapamycin MP treated group FIG. 14f), and among these cells a higher frequency of T_{REG} (FIG. 14g). There were no differences in CD44⁺ CD62L⁺ expression (FIG. 14h, 14i.) among all antigen-specific T_{REG}, which is to be expected since CD62L drives entry of T cells into LNs, and activated CD44⁺ T cells not expressing CD62L are restricted to circulation and residence in tissue. Excitingly, however, BCL2 (FIG. 14j) and CD127 (FIG. 14k) expression was significantly higher among antigen-specific T_{REG} in CgA/rapamycin MP treated LNs compared to CgA MP treated LNs. In summary, CgA/rapamycin MP treatment expanded antigen-specific T_{REG} in LNs which displayed increased expression of markers associated with cell durability.

MP treatment promotes expression of memory markers among antigen-specific T_{REG} and durable tolerance in T1D. BDC2.5 T cell co-cultures were treated with CgA MPs or CgA/rapamycin MPs. (FIG. 14a) representative gating scheme to quantify co-expression of CD44 and CD621 among BDC2.5 T_{REG}. Quantification of (FIG. 14b) co-expression of CD44 and CD62L among BDC2.5 T_{REG} and (FIG. 14d) mean fluorescence intensity of CCR7 among BDC2.5 T_{REG}. N=3 for each sample. One way ANOVA with Tukey's post hoc testing was used to compare all groups. Comparisons for CgA MP and CgA/rapamycin MPs at matched doses are indicated in plots. (FIG. 14e) Schematic representation of BDC2.5 T cell adoptive transfer experiment in FIG. 12f-k. N=5 for each treatment group. (FIG. 14f) Representative flow cytometry plots showing BDC2.5 T cell frequency in treated LNs. Gating scheme for (FIG. 14g) CgA specific T_{REG} and (FIG. 14h) co-expression of CD44 and CD621, and expression of BCL-2 and CD127 among CgA specific T_{REG}. Quantification of (FIG. 14i) co-expression of CD44 and CD621, and expression of (FIG. 14j) BCL-2 and (FIG. 14k) CD127 among CgA specific T_{REG} in treated LNs. Treated LNs were pooled for each mouse. Two tailed Mann Whitney testing was used to compare CgA MP vs CgA/rapamycin MP treatment groups. (FIG. 14l) Schematic representation of NRP-V7 adoptive transfer induced T1D experiment. N=8 for each treatment group. The number of surviving mice in each treatment group is shown in parenthesis in the legend. (FIG. 14m) Survival curve indicating percent of mice remaining normoglycemic. Two tailed log-rank (Mantel-Cox) was used for all pairwise comparisons between each treatment.

MP treatment provides durable immunosuppression in the NRP-V7 model of T1D. We tested whether the tolerance induced by MP treatment would be maintained even if T1D induction was performed at a much later timepoint after treatment with the MPs. NOD mice were immunized 10 days before infusion of activated NRP-V7 T cells, and disease incidence was monitored daily (FIG. 14l). Excitingly CgA/rapamycin MPs and NRP-V7/rapamycin MPs significantly improved survival relative to the vehicle and CgA MP treated mice. Although significance was not achieved when comparing CgA/rapamycin MPs and NRP-V7/rapamycin MPs to rapamycin MP treatment (p=0.1870 and p=0.0655), the frequency of surviving mice was more than doubled for both formulations combining antigen and rapamycin. Taken together, FIG. 14 indicates LN treatment of MPs co-encapsulating islet antigen and rapamycin expands antigen-specific T_{REG} with a memory-like phenotype and promotes durable tolerance in T1D.

There are several possible explanations for increased expansion of CgA-specific T cells after treatment with CgA/rapamycin MP compared to CgA MP (FIG. 10g; FIG. 14f). Antigen-specific T cell proliferation may be more sustained after CgA/rapamycin MP treatment. Additionally, reduced mTOR signaling during antigenic stimulation of T cells may drive a more durable phenotype. Previous reports have established mTOR inhibition to promote central memory phenotypes among cytotoxic T cells however (Araki, K. et al. (2009) mTOR regulates memory CD8 T-cell differentiation. Nature 460, 108-112; Gattinoni, L., Klebanoff, C. A. & Restifo, N. P. (2009) Pharmacologic Induction of CD8+ T Cell Memory: Better Living Through Chemistry. Sci. Transl. Med. 1, 11ps12; Joshua M. Gammon et al. (2017) Low-dose controlled release of mTOR inhibitors maintains T cell plasticity and promotes central memory T cells. J. Controlled Release).

However, only recently have the effects of mTOR inhibition on T_{REG} memory phenotype been explored (see, e.g., Smigiel, K. S. et al. (2013) CCR7provides localized access to IL-2 and defines homeostatically distinct regulatory T cell subsets. J. Exp. Med. 211, 121-136). Studies have demonstrated T_{REG} in lymphoid tissue with low mTOR activity display increased markers of memory phenotype (BCL2^{high}, CD62L^{high}, KLRG1^{low}, PD-1^{low}), lower levels of aerobic glycolysis, higher levels of oxidative phosphorylation, and prolonged survival—all characteristics of long lived memory T cells (Sun, I.-H. et al. (2018) mTOR Complex 1 Signaling Regulates the Generation and Function of Central and Effector Foxp3+ Regulatory T Cells. J. Immunol. 201(2):481-492). While studies have investigated co-delivering mTOR inhibitors with antigen to promote antigen-specific T_{REG} (Maldonado, R. A. et al. (2015) Polymeric synthetic nanoparticles for the induction of antigen specific immunological tolerance. Proc. Natl. Acad. Sci. 112, E156-E165; Tostanoski, L. H. et al. (2016) Reprogramming the Local Lymph Node Microenvironment Promotes Tolerance that Is Systemic and Antigen Specific. Cell Rep. 16, 2940-2952; LaMothe, R. A. et al. (2018) Tolerogenic Nanoparticles Induce Antigen-Specific Regulatory T Cells and Provide Therapeutic Efficacy and Transferrable Tolerance against Experimental Autoimmune Encephalomyelitis. Front. Immunol. 9, 281), the present disclosure is the first to investigate the memory phenotype generated using this strategy. Here, CgA/rapamycin MP induced antigen-specific T_{REG} expressed higher levels of markers associated with longevity (FIG. 14h, 14j, 14k), and CgA/rapamycin MP treatment provided superior protection from T1D when disease was induced at a late timepoint following treatment (FIG. 14m). Together these data demonstrate that intra-LN treatment of tolerogenic MPs in accordance with disclosed embodiments promote a durable and functional antigen-specific T_{REG} response.

Thus, the intra-LN platform of the present disclosure may be utilized to evaluate and identify selected immune signals that promote tolerance, and integrate these signals in the LN modulate antigen-specific T cell response. These results demonstrated potent expansion of antigen-specific T_{REG} in treated and untreated LNs after intra-LN treatment with CgA/rapamycin MP, thus providing the strongest therapeutic benefit in T1D and graft transplant rejection. While the expanded non-T_{REG} cells may provide pro-inflammatory response, it is clear they are controlled in vivo. In accordance with the present disclosure, a single LN treatment promoted tolerance in a model of T1D and suppression in a model of islet transplantation (FIG. 15) thus demonstrating suppression of adverse inflammatory response against pancreatic islets.

As demonstrated by the disclosed data, the direct LN injection of MPs co-encapsulating rapamycin with selected antigens targeted in autoimmune disease and alloimmune response is a potent therapeutic strategy to promote antigen-specific tolerance. MP treatment dramatically increased survival in models of T1D and islet transplantation mediated by both CD4 or CD8 pathology (FIG. 15). Co-encapsulation of both MP components is beneficial for optimal therapeutic efficacy. Mechanistically, inclusion of antigen primes antigen-specific T cells in LNs, while rapamycin functions to inhibit mTOR signaling during T cell priming. Excitingly, MPs expand T1D-relevant antigen-specific T_{REG} in treated as well as distant LNs, creating tolerogenic microdomains in LNs that promote these regulatory immune responses. Further, MP treatment induced expression of markers associated with prolonged persistence and memory function among antigen-specific T_{REG} and promoted durable tolerance in T1D. Together these data demonstrate a modular platform that effectively promotes lasting and beneficially selective tolerance in the context of T1D, and with reduced or eliminated adverse side effects given systemic or peripheral delivery is not required.

Concluding, local lymph node delivery of tolerogenic microparticles encapsulating autoantigens and an immune modulatory agent (e.g., rapamycin) promote tolerance in a model of T1D as well as a model of islet transplantation. In islet transplantation, microparticle treatment promoted tolerogenic zones in LNs characterized by increases in laminin α4:α5 ratios and antigen specific T_{REG} accumulation in HEVs and CRs. Lymph node delivery of tolerogenic microparticles promoted tolerance in T1D through remodeling of the lymph node microenvironment to promote long lasting antigen specific regulatory T cells. Local LN remodeling correlates with increased antigen specific T_{REG} and reduced T cell activation in grafts. In spontaneous T1D, antigen specific T cells were primed early in treated LNs with low mTOR activity. At later timepoints, antigen specific T cells were expanded and activated in distant LNs and are polarized toward T_{REG}. It is believed that these mechanisms are occurring in both models of antigen specific tolerance. Further, it is believed that tolerogenic microparticles promote enhanced survival and memory phenotype in antigen specific T cells, through suppressing mTOR during priming and expansion.

All identified publications and references mentioned in this specification are herein incorporated by reference to the same extent as if each individual publication was specifically and individually indicated to be incorporated by reference in its entirety. While the disclosure has been described in connection with exemplary embodiments thereof, it will be understood that it is capable of further modifications, and this application is intended to cover any variations, uses, or adaptations of the disclosure following, in general, the principles of the disclosure and including such departures from the present disclosure as come within known or customary practice within the art to which the disclosure pertains and as may be applied to the features hereinbefore set forth.

What is claimed is:

1. A method of promoting immune tolerance to an antigen in a subject, said method comprising introducing directly into at least one lymph node of the subject:
(a) a therapeutically effective amount of a composition comprising an antigen associated with an autoimmune disease or disorder, in combination with
(b) a carrier comprising an immune modulatory agent such that an immune response to said antigen is inhibited or suppressed in the subject.

2. The method of claim 1, wherein said carrier is a microparticle.

3. The method of claim 2, wherein said microparticle has a diameter of between about 1 μm and about 10 μm.

4. The method of any one of claims 2-3, wherein said microparticle is biodegradable and/or biocompatible.

5. The method of any one of claims 2-4, wherein said microparticle comprises a polymer material selected from the group consisting of poly(glycolide) (PGA), poly(L-lactide) (PLA), poly(beta-amino esters), and polyethylene glycol (PEG).

6. The method of any one of claims 1-5, wherein said antigen and/or said immune modulatory agent is introduced directly into the at least one lymph node of the subject via intra-lymph node injection.

7. The method of any one of claims 1-6, wherein said immune modulatory agent is selected from the group consisting of a calcineurin inhibitor, an interleukin inhibitor, a TNF alpha inhibitor, and a selective immunosuppressant.

8. The method of claim 7, wherein said immune modulatory agent is an mTOR inhibitor.

9. The method of claim 8, wherein said mTOR inhibitor is rapamycin or derivative thereof.

10. The method of any one of claims 1-9, wherein said antigen and said immune modulatory agent are introduced into a single lymph node of the subject.

11. The method of any one of claims 1-9, wherein said antigen and said immune modulatory agent are introduced into at least two lymph nodes of the subject.

12. The method of any one of claims 1-9, wherein said antigen is introduced into one or more lymph node(s) of the subject, and said immune modulatory agent is introduced into one or more lymph node(s) of the subject different from the one or more lymph node(s) into which said antigen is introduced.

13. The method of any one of claims 1-12, wherein said autoimmune disease or condition is type 1 diabetes.

14. The method of any one of claims 1-12, wherein said autoimmune disease or condition is associated with a graft transplant.

15. The method of any one of claims 1-14, wherein said antigen is an islet antigen.

16. The method of any one of claims 1-15, wherein said antigen is selected from the group consisting of: chromogranin A (CgA); NRP-V7; insulin; GAD65; GAD67; carboxypeptidase H; insulinoma associated antigen-2 (IA-2); insulinoma associated antigen-2 beta (IA-2β; also termed phogrin or ICA512); imogen 38; islet-specific glucose-6-phosphatase catalytic subunit-related protein (IGRP); ZnT8; islet amyloid polypeptide (IAPP); Tetraspanin-7 (Tspan7; P4Hb; GRP78: Urocortin-3; and MHC antigen class I and class II molecules (including Eα and Eβ).

17. The method of any one of claims 1-16, wherein the subject is a human.

18. A method of treating or preventing an autoimmune disease or condition in a subject in need thereof, comprising introducing directly into at least one lymph node of the subject:
(a) a therapeutically effective amount of a composition comprising an antigen associated with an autoimmune disease or disorder, in combination with
(b) a carrier comprising an immune modulatory agent such that an immune response to said antigen is inhibited or suppressed in the subject.

19. The method of claim 18, wherein said carrier is a microparticle.

20. The method of claim 19, wherein said microparticle has a diameter of between about 1 μm and about 10 μm.

21. The method of any one of claims 18-20, wherein said microparticle is biodegradable and/or biocompatible.

22. The method of any one of claims 19-21, wherein said microparticle comprises a polymer material selected from the group consisting of poly(glycolide) (PGA), poly(L-lactide) (PLA), poly(beta-amino esters), and polyethylene glycol (PEG).

23. The method of any one of claims 18-22, wherein said antigen and/or said immune modulatory agent is introduced directly into the at least one lymph node of the subject via intra-lymph node injection.

24. The method of any one of claims 18-23, wherein said immune modulatory agent is selected from the group consisting of a calcineurin inhibitor, an interleukin inhibitor, a TNF alpha inhibitor, and a selective immunosuppressant.

25. The method of claim 24, wherein said immune modulatory agent is an mTOR inhibitor.

26. The method of claim 25, wherein said mTOR inhibitor is rapamycin or a rapamycin derivative.

27. The method of any one of claims 18-26, wherein said antigen and said immune modulatory agent are introduced into a single lymph node of the subject.

28. The method of any one of claims 18-26, wherein said antigen and said immune modulatory agent are introduced into at least two lymph nodes of the subject.

29. The method of any one of claims 18-26, wherein said antigen is introduced into one or more lymph node(s) of the subject, and said immune modulatory agent is introduced into one or more lymph node(s) of the subject different from the one or more lymph node(s) into which said antigen is introduced.

30. The method of any one of claims 18-29, wherein said autoimmune disease or condition is type 1 diabetes.

31. The method of any one of claims 18-29, wherein said autoimmune disease or condition is associated with a graft transplant.

32. The method of any one of claims 18-31, wherein said antigen is an islet antigen.

33. The method of any one of claims 18-32, wherein said antigen is selected from the group consisting of: chromogranin A (CgA); NRP-V7; insulin; GAD65; GAD67; carboxypeptidase H; insulinoma associated antigen-2 (IA-2); insulinoma associated antigen-2 beta (IA-2β; also termed phogrin or ICA512); imogen 38; islet-specific glucose-6-phosphatase catalytic subunit-related protein (IGRP); ZnT8; islet amyloid polypeptide (LAPP): Tetraspanin-7 (Tspan7); P411b; GRP78; Urocortin-3; and MHC antigen class I and class II molecules (including Eα and Eβ).

34. The method of any one of claims 18-33, wherein the subject is a human.

35. A pharmaceutical composition comprising a therapeutically effective amount of an antigen associated with an autoimmune disease or disorder, an immune modulatory agent formulated to inhibit or suppress an immune response to the antigen in a subject, and a pharmaceutically acceptable carrier, diluent, and/or excipient.

36. The pharmaceutical composition of claim 35, wherein said carrier is a microparticle.

37. The pharmaceutical composition of claim 36, wherein said microparticle has a diameter of between about 1 μm and about 10 μm.

38. The pharmaceutical composition of any one of claims 35-37, wherein said microparticle comprises a polymer material selected from the group consisting of poly(glycolide) (PGA), poly(L-lactide) (PLA), poly(beta-amino esters), and polyethylene glycol (PEG).

39. The pharmaceutical composition of any one of claims 35-38, which is formulated for administration via intra-lymph node injection.

40. The pharmaceutical composition of any one of claims 35-39, wherein said immune modulatory agent is selected from the group consisting of a calcineurin inhibitor, an interleukin inhibitor, a TNF alpha inhibitor, and a selective immunosuppressant.

41. The pharmaceutical composition of claim 40, wherein said immune modulatory agent is an mTOR inhibitor.

42. The pharmaceutical composition of claim 41, wherein said mTOR inhibitor is rapamycin or derivative thereof.

43. The pharmaceutical composition of any one of claims 35-42, wherein said autoimmune disease or condition is type 1 diabetes.

44. The pharmaceutical composition of any one of claims 35-42, wherein said autoimmune disease or condition is associated with a graft transplant.

45. The pharmaceutical composition of any one of claims 35-44, wherein said antigen is an islet antigen.

46. The pharmaceutical composition of any one of claims 35-45, wherein said antigen is selected from the group consisting of: chromogranin A (CgA); NRP-V7; insulin; GAD65; GAD67; carboxypeptidase H; insulinoma associated antigen-2 (IA-2); insulinoma associated antigen-2 beta (IA-2β; also termed phogrin or ICA512); imogen 38; islet-specific glucose-6-phosphatase catalytic subunit-related protein (IGRP); ZnT8; islet amyloid polypeptide (IAPP); Tetraspanin-7 (Tspan7); P4Hb; GRP78; Urocortin-3; and MHC antigen class I and class II molecules (including Eα and Eβ).
